(19) United States
(12) Patent Application Publication
(10) Pub. No.: US 2014/0237732 A1
(43) Pub. Date: Aug. 28, 2014

(54) HAIR CARE AGENTS WITH SELECTED NON-IONIC POLYMERS AND CATIONIC SILICONES

(71) Applicant: Henkel AG & Co. KGaA, Duesseldorf (DE)

(72) Inventors: Nicole Zuedel Fernandes, Hamburg (DE); Marlene Battermann, Asendorf (DE)

(73) Assignee: Henkel AG & Co. KGaA, Duesseldorf (DE)

(21) Appl. No.: 14/278,355

(22) Filed: May 15, 2014

Related U.S. Application Data
(63) Continuation of application No. PCT/EP2012/069978, filed on Oct. 9, 2012.

(30) Foreign Application Priority Data
Nov. 18, 2011 (DE) 10 2011 086 632.9

(51) Int. Cl. A61K 8/49; A61K 8/33; A61K 8/25; A61Q 5/10

(52) U.S. Cl. CPC A61K 8/4913; A61Q 5/10; A61K 8/4906; A61K 8/33; A61K 8/25
USPC 8/405

(57) ABSTRACT

Foaming hair treatment agents including selected non-ionic polymers and cationic silicones. In modern hair care agents the aim increasingly is to apply the product to the keratinic fibers in the form of a foam. The foam should be creamy and fine-pored, and at the same time should have a certain volume and be stable enough to remain stable while it is worked into the keratinic fibers.

### FIELD OF THE INVENTION

The present invention generally relates to hair treatment agents including selected non-ionic polymers and cationic silicones.

### BACKGROUND OF THE INVENTION

In modern hair care agents the aim increasingly is to apply the product to the keratinic fibers in the form of a foam. The foam should be creamy and fine-pored, and at the same time should have a certain volume and be stable enough to remain stable while it is worked into the keratinic fibers. Until now these requirements could be met only with large amounts of surfactants and/or propellants. This type of application is even more difficult if no propellant is to be used and the foam is to be produced instead in pump dispensers by means of special nozzles. Frequently only relatively aqueous, large-pored and unstable foams are obtained in such cases. The more caring ingredients that are include d in the aqueous composition, the greater a problem this is.

Environmental influences and oxidative hair treatments often lead to poorer combability of the dry and wet hair. Furthermore, the shine and moisture balance are adversely influenced by the attack on the external structure of the keratinic fibers. A further consequence of repeated treatments of keratinic fibers with surfactant and/or oxidative agents is a high degree of grease buildup in the keratinic fibers as well as a strong tendency to the increased formation of dandruff.

An object of the present invention is therefore to prevent the disadvantages of the prior art as described above. In addition to producing a fine-pored, creamy and stable foam, side-effects of environmental influences and of oxidative and surfactant hair treatments should be reduced, preferably during the actual oxidative or surfactant hair treatment but also after the oxidative or surfactant hair treatment, without adversely affecting the efficiency of the oxidative or surfactant cosmetic, in particular with regard to color intensity, color fastness, lightening capacity or waving effect, and to prevent grease buildup on the keratinic fibers and the increased formation of dandruff. Furthermore, even in the form of a 2-in-1 product, the oxidative treatment of keratin-including fibers, in particular human hair, should be combined in one application step with the application of an effective protection of the fibers against environmental influences, for example a UV protection.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair treatment agent including in a suitable cosmetic carrier—relative in each case to the total composition of the agent—a) at least one non-ionic polymer in a total amount from 0.01 to 10.0 wt. %, selected from the non-ionic polymers including at least one structural unit selected from the group of structural units of formulae (M1) to (M3)

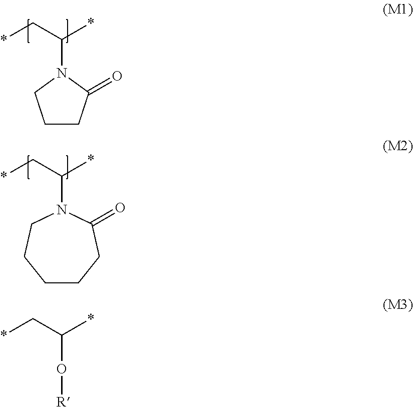

in which R′ denotes a hydrogen atom or a (C₂ to C₁₈) acyl group; and b) at least one cationic amino silicone having at least three terminal amino-functional groups in a total amount from 0.01 to 5.0 wt. %.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly it has now been found that the object is achieved to an outstanding degree by a hair treatment agent which includes an active ingredient complex including as essential ingredients at least one selected non-ionic polymer and at least one cationic silicone having at least three terminal amino-functional groups.

Hair treatment agents within the meaning of the present invention are for example hair shampoos, hair conditioners, conditioning shampoos, hair rinses, hair masks, hair packs, hair tonics, hair coloring shampoos or combinations thereof. In particular, the hair treatment agents according to the invention are understood to be hair conditioning compositions such as hair rinses, hair masks, hair packs, hair oils and hair lotions, both as leave-on products, i.e. products that remain on the hair until the next time it is washed, and as rinse-off products, i.e. products that are rinsed out again a few seconds up to a few hours after application.

According to the invention combability is understood to mean both the combability of wet fibers and the combability of dry fibers.

Feel is defined as the tactility of a group of fibers, the person skilled in the art feeling and assessing the parameters fullness and softness of the group of fibers by sensory means.

Shaping is understood to be the ability to change the shape of a group of previously treated keratin-including fibers, in particular human hair. In hair cosmetics this is also referred to as styling ability.

Restructuring within the meaning of the invention is understood to be a reduction in the damage that is caused to keratinic fibers by a very wide variety of influences. The restoration of natural strength, for example, plays an important part in this.

Restructured fibers are characterized by an improved shine, an improved feel and easier combability. They also have improved strength and elasticity. Furthermore, a successful restructuring can be physically detected as an increase in the melting point in comparison to damaged fibers. The higher the melting point of the hair, the stronger the structure of the fiber.

Wash fastness within the meaning of the invention is understood to be the retention of the shade and/or intensity of the original color when the keratinic fiber is exposed to the repeated influence of aqueous agents, in particular surfactant-including agents such as shampoos.

Film-forming polymers are understood to be polymers which leave behind a continuous film on the skin, hair or nails when they dry. Such film formers can be used in a wide range of cosmetic products, such as for example face masks, make-up, hair fixing agents, hair sprays, hair gels, hair waxes, hair masks, shampoos or nail varnishes. Such polymers are preferred in particular which have an adequate solubility in water or water/alcohol mixtures in order for them to be present in the agent according to the invention in completely dissolved form. The film-forming polymers can be of synthetic or natural origin.

Film-forming polymers are also understood according to the invention to be polymers which when used in a 0.01 to 20 wt. % aqueous, alcoholic or aqueous-alcoholic solution are capable of depositing a transparent polymer film on the hair.

Fixing polymers contribute to the hold and/or to establishing volume and fullness in the hairstyle as a whole. These polymers are simultaneously also film-forming polymers and are therefore generally typical substances for hair treatment agents such as hair fixing agents, hair foams, hair waxes, hair sprays. Film formation may be entirely localized here and may bond only a few fibers together.

The curl retention test is frequently used as a test method for the fixing effect of a polymer.

These hair treatment agents according to the invention produce a voluminous, fine-pored and creamy, stable foam, which also makes stable foams in particular in non-aerosols such as pump dispenser systems. Furthermore, hair treatment agents including this active ingredient complex lead to an improvement in finish, to an improvement in shine, to an improvement in moisture balance and to protection from oxidative damage and a prevention of grease buildup in the keratinic fibers as well as to an increase in the washing resistance of colored keratinic fibers, in particular human hair, and to a temporal delay in the formation of dandruff.

The compositions according to the invention including the active ingredient complex according to the invention are characterized moreover by a markedly improved condition of the keratinic fibers in terms of the moisture balance of the keratinic fibers. Furthermore, the active ingredient complex according to the invention leads to a marked protection of the keratinic fibers against the effects of heat, for example when blow-drying keratinic fibers. Protecting the surface of keratinic fibers against the effects of heat is very important, particularly when using hair straighteners or hair dryers. Finally, it was established that the compositions according to the invention surprisingly lead to a marked lengthening of the time taken for the keratinic fibers to become dirty again

To determine the foam qualities the composition according to the invention is fully expanded, and to determine the stability the time taken for the foam to start to change back into a purely aqueous composition is measured.

The volume of the compositions is measured at the same time as they are expanded. The higher this volume, the better the composition in terms of volume.

Immediately after it is expanded, the creaminess of the foam is assessed by trained persons by sensory means. Furthermore, the pore size is assessed both subjectively and objectively and also measured.

An aqueous cosmetic carrier includes at least 50 wt. % of water.

Within the meaning of the present invention aqueous-alcoholic cosmetic carriers are understood to be aqueous solutions including 3 to 70 wt. % of a C₁-C₆ alcohol, in particular methanol, ethanol or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol or 1,6-hexanediol. The agents according to the invention can additionally include further organic solvents, such as for example methoxybutanol, benzyl alcohol, ethyl diglycol or 1,2-propylene glycol. All water-soluble organic solvents are preferred here. Water is particularly preferred.

The present invention therefore firstly provides a hair treatment agent including in a suitable cosmetic carrier—relative in each case to the total composition of the agent—
a) at least one non-ionic polymer in a total amount from 0.01 to 10.0 wt. %, selected from the non-ionic polymers including at least one structural unit selected from the group of structural units of formulae (M1) to (M3)

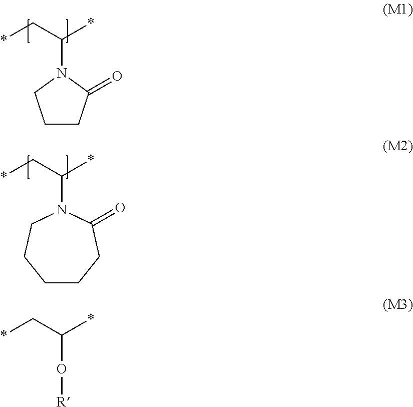

in which R′ denotes a hydrogen atom or a (C₂ to C₁₈) acyl group

and
b) at least one cationic amino silicone having at least three terminal amino-functional groups in a total amount from 0.01 to 5.0 wt. %.

The use of this combination leads to surprisingly good properties in the treated hair, in particular to improved combability, to improved shine and to an improved elasticity as well as to a marked increase in the washing resistance of colored hair and to a longer hold combined at the same time with a better shaping performance in waving processes such as finger waving and permanent waving. In particular, however, a high foam volume with a fine-pored and very creamy foam is obtained, which is very stable over the application period.

The first required ingredient, the non-ionic polymer, is a non-ionic polymer including at least one structural unit selected from the group of structural units of formulae (M1) to (M3)

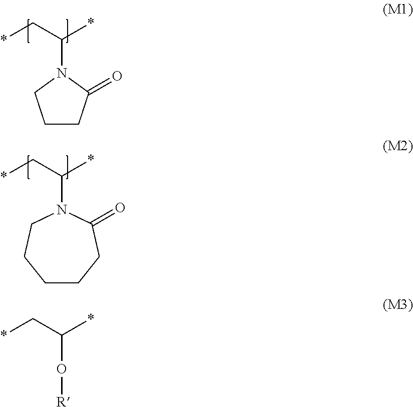

in which R′ denotes a hydrogen atom or a (C₂ to C₁₈) acyl group.

In the above formulae and all subsequent formulae, a chemical bond marked with the symbol * denotes a free valence of the corresponding structural fragment.

The properties of the composition according to the invention prove to be particularly advantageous if it is presented as an aerosol spray, aerosol foam, pump spray or pump foam. This preferred presentation form is described in detail further on.

A non-ionic polymer is understood according to the invention to be a polymer which in a protic solvent under standard conditions substantially bears no structural units having cationic or anionic groups needing to be offset by counterions to maintain electrical neutrality. Cationic groups include for example quaternized ammonium groups and protonated amines. Anionic groups include for example carboxyl and sulfonic acid groups.

The non-ionic polymers a) are preferably include d in the compositions according to the invention in an amount from 0.01 wt. % to 10.0 wt. % particularly preferably from 0.1 wt. % to 10.0 wt. %, most particularly preferably from 0.1 wt. % to 5.0 wt. %, relative in each case to the weight of the agent according to the invention.

According to the invention, non-ionic polymers a) having at least one structural element of formula (M3) which according to formula (M3) bear a hydrogen atom, an acetyl group or a propanoyl group, in particular a hydrogen atom or an acetyl group, as R′, are preferably suitable.

The non-ionic polymers a) are preferably selected in turn from at least one polymer of the group formed from

homopolymers and non-ionic copolymers of N-vinylpyrrolidone,

polyvinyl alcohol,

polyvinyl acetate.

Suitable polyvinylpyrrolidones are for example commercial products such as Luviskol® K 90 or Luviskol® K 85 from BASF SE.

Suitable polyvinyl alcohols are sold for example under the trade names Elvanol® by Du Pont or Vinol® 523/540 by Air Products.

Suitable polyvinyl acetate is sold for example as an emulsion under the trade name Vinac® by Air Products.

Compositions including as the non-ionic polymer a) at least one polymer selected from the group formed from
polyvinylpyrrolidone,
copolymers of N-vinylpyrrolidone and vinyl esters of carboxylic acids having 2 to 18 carbon atoms, in particular of N-vinylpyrrolidone and vinyl acetate,
   are most particularly preferred according to the invention.

In the context of this embodiment, agents that are most particularly preferred are furthermore in particular those which in a cosmetically acceptable carrier include as the non-ionic polymer:

polyvinylpyrrolidone.

In the context of this embodiment, agents that are most particularly preferred are furthermore in particular those which in a cosmetically acceptable carrier include as the non-ionic polymer:
a) a copolymer produced from the monomers N-vinylpyrrolidone and vinyl acetate and in particular from no further monomer.

It is in turn preferable here for the molar ratio in the polymer of structural units include d from the N-vinylpyrrolidone monomer to structural units include d from the vinyl acetate monomer to be in the range from 20 to 80 to 80 to 20, in particular from 30 to 70 to 60 to 40.

Suitable copolymers of vinylpyrrolidone and vinyl acetate are obtainable for example from BASF SE under the trademarks Luviskol® VA 37, Luviskol® VA 55, Luviskol® VA 64 and Luviskol® VA 73.

In addition to the non-ionic polymer a) that is used, the agents according to the invention can also include at least one further non-ionic polymer that is different from polymer a).

The second required component of the active ingredient complex is a cationic amino silicone. Cationic amino silicones having at least three terminal amino-functional groups have only recently become commercially available. These cationic silicone polymers are characterized in that they have a silicone framework and optionally a polyether part and moreover at least one part having an ammonium structure. Examples of the preferred cationic silicone polymers within the meaning of the present invention are in particular the compounds with the INCI names: Silicone Quatemium-1, Silicone Quaternium-2, Silicone Quaternium-3, Silicone Quaternium-4, Silicone Quatemium-5, Silicone Quatemium-6, Silicone Quaternium-7, Silicone Quaternium-8, Silicone Quaternium-9, Silicone Quatemium-10, Silicone Quatemium-11, Silicone Quatemium-12, Silicone Quaternium-15, Silicone Quatemium-16, Silicone Quatemium-17, Silicone Quatemium-18, Silicone Quatemium-20, Silicone Quaternium-21, Silicone Quaternium-22 as well as Silicone Quaternium-2 Panthenol Succinate and Silicone Quatemium-16/Glycidyl Dimethicone Crosspolymer. Silicone Quaternium-22 is most preferred in particular. This raw material is sold for example by Evonik under the trade name Abil® T-Quat 60.

The cationic amino-functional silicone polymers are include d in the compositions according to the invention in amounts from 0.01 to 5 wt. %, preferably in amounts from 0.05 to 5 wt. % and most particularly preferably in amounts from 0.1 to 5 wt. %. The very best results are obtained with amounts from 0.1 to 2.5 wt. %, relative in each case to the total composition of the individual agent.

To intensify the effect according to the invention the agents according to the invention preferably additionally include at least one surfactant, with non-ionic, anionic, cationic and ampholytic surfactants being suitable in principle. According to the invention the surfactants can already have an emulsifying action. The group of ampholytic or amphoteric surfactants comprises zwitterionic surfactants and ampholytes and is highly preferred according to the invention.

In the compositions according to the invention these ingredients possibly make a considerable contribution to stabilizing the viscosity and the storage properties.

Particularly suitable zwitterionic surfactants are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytic surfactants (Tampho) are understood to be surface-active compounds that are capable of forming internal salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Typical examples of amphoteric or zwitterionic surfactants are alkyl betaines, alkyl amido betaines, aminopropionates, aminoglycinates, imidazolinium betaines and sulfobetaines. Particularly preferred ampholytic surfactants are N-cocoalkyl aminopropionate, cocoacylaminoethyl aminopropionate and C₁₂-C₁₈ acyl sarcosine. Coco betaine is a particularly preferred compound.

These ingredients are used in amounts from 0.01 to 5.0 wt. %, relative to the total composition of the agent. Amounts from 0.05 to 5.0 wt. % are preferred. Amounts from 0.1 to 5.0 wt. % are particularly preferred, highly preferably from 0.3 to 3.0 wt. %.

The present invention therefore secondly provides a hair treatment agent including in a suitable cosmetic carrier—relative in each case to the total weight of the composition of the agent—
a) at least one non-ionic polymer in a total amount from 0.01 to 10.0 wt. %, selected from the non-ionic polymers including at least one structural unit selected from the group of structural units of formulae (M1) to (M3)

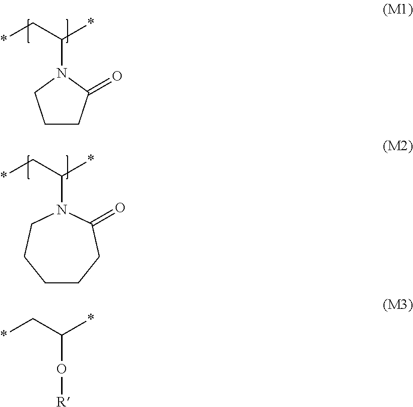

in which R′ denotes a hydrogen atom or a (C₂ to C₁₈) acyl group,

and
b) at least one cationic amino silicone having at least three terminal amino-functional groups in a total amount from 0.01 to 5.0 wt. %, and
c) at least one surfactant selected from the zwitterionic and/or amphoteric surfactants in a total amount from 0.01 to 5.0 wt. %.

It is most particularly preferred according to the invention if the compositions according to the invention moreover include at least one quaternary compound. The effectiveness of the agent according to the invention is further increased in this way and the stability of the composition is boosted considerably.

Cationic compounds are quaternary ammonium compounds. In principle these are monomeric cationic or amphoteric ammonium compounds, monomeric amines, amino amides, polymeric cationic ammonium compounds and polymeric amphoteric ammonium compounds. Of this large number of possible quaternary ammonium compounds, the following cationic compounds have proved particularly suitable.

Esterquats according to the formula (Tkat1-2) are the first group of particularly preferred cationic compounds.

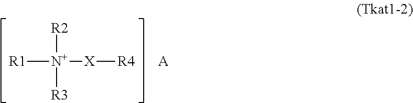

Here residues R1, R2 and R3 are each independent of one another and can be identical or different. Residues R1, R2 and R3 denote:
a branched or unbranched alkyl residue having 1 to 4 carbon atoms, which can include at least one hydroxyl group, or
a saturated or unsaturated, branched or unbranched or a cyclic saturated or unsaturated alkyl residue having 6 to 30 carbon atoms, which can include at least one hydroxyl group, or
an aryl or alkaryl residue, for example phenyl or benzyl,
the residue (—X—R4), with the proviso that at most 2 of residues R1, R2 or R3 can denote this residue.

The residue —(X—R4) is include d at least 1 to 3 times.

Here X denotes:
1) —(CH2)n- where n=1 to 20, preferably n=1 to 10 and particularly preferably n=1 to 5, or
2) —(CH2-CHR5-O)n- where n=1 to 200, preferably 1 to 100, particularly preferably 1 to 50, and particularly preferably 1 to 20 with R5 denoting hydrogen, methyl or ethyl,
3) a hydroxyalkyl group having one to four carbon atoms, which can be branched or unbranched and which can include at least one and at most three hydroxyl groups. Examples of —X— are: —CHOH, —CHCH₂OH, —CH₂CHOH, —COHCHOH, —CHOHCOH, —CHCHOHCH₃, —CH₂COHCH₃, —CH₂CHOHCH₂—, —C(CH₂OH)₂, —CH₂CHOHCH₂OH, —CH₂CH₂CHOH, —CH₂COHCH₃ and hydroxybutyl residues, the binding of —X— to R4 starting from the free valence of the corresponding carbon atom and R4 denotes:
1) R6-O—CO—, in which R6 is a saturated or unsaturated, branched or unbranched or a cyclic saturated or unsaturated alkyl residue having 6 to 30 carbon atoms, which can include at least one hydroxyl group and which can optionally also be ethoxylated with 1 to 100 ethylene oxide units and/or 1 to 100 propylene oxide units, or
2) R7-CO—, in which R7 is a saturated or unsaturated, branched or unbranched or a cyclic saturated or unsaturated alkyl residue having 6 to 30 carbon atoms, which can include at least one hydroxyl group and which can optionally also be ethoxylated with 1 to 100 ethylene oxide units and/or 1 to 100 propylene oxide units,
   and A denotes a physiologically acceptable organic or inorganic anion and is defined here also as a representative of all structures described below. The anion of all cationic compounds that are described here is selected from the halide ions, fluoride, chloride, bromide, sulfates of the general formula RSO₃⁻, in which R has the meaning of saturated or unsaturated alkyl residues having 1 to 4 carbon atoms, or anionic residues of organic acids such as maleate, fumarate, oxalate, tartrate, citrate, lactate or acetate. Such products are sold under the trademarks Rewoquat®, Stepantex®, Dehyquart®, Armocare® and Akypoquat® for example. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131 are examples of these esterquats.

Further compounds of formula (Tkat1-2) that are particularly preferred according to the invention are include d in formula (Tkat1-2.1), cationic betaine esters.

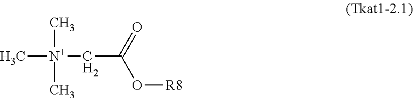

R8 corresponds in its meaning to R7.

The esterquats with the trade names Armocare® VGH-70 and also Dehyquart® F-75, Dehyquart® L80, Stepantex® VS 90 and Akypoquat® 131 are particularly preferred.

Quaternary imidazoline compounds are a further group. Formula (Tkat2) below shows the structure of these compounds.

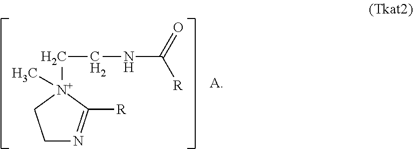

The residues R independently of one another each denote a saturated or unsaturated, linear or branched hydrocarbon residue having a chain length of 8 to 30 carbon atoms. The preferred compounds of formula (Tkat2) each include the same hydrocarbon residue for R. The chain length of the residues R is preferably 12 to 21 carbon atoms. A denotes an anion as described above. Particularly suitable examples according to the invention are available for example under the INCI names Quatemium-27, Quaternium-72, Quaternium-83 and Quatemium-91. Quatemium-91 is highly preferred according to the invention.

In a particularly preferred embodiment of the invention the agents according to the invention include furthermore at least one amine and/or cationized amine, in particular an amido amine and/or a cationized amido amine of the following structural formulae:

R1-NH—(CH₂)ₙ—N⁺R²R³R⁴A (Tkat3)

in which R¹ denotes an acyl or alkyl residue having 6 to 30 C atoms, which can be branched or unbranched, saturated or unsaturated, and wherein the acyl residue and/or alkyl residue can include at least one OH group, and
R2, R3 and R4 in each case independently of one another denote
1) hydrogen or
2) an alkyl residue having 1 to 4 C atoms, which can be the same or different, saturated or unsaturated, and
3) a branched or unbranched hydroxyalkyl group having 1 to 4 carbon atoms with at least one and at most three hydroxyl groups, for example —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues, and
   A denotes an anion as described above, and
   n denotes an integer between 1 and 10.

A composition in which the amine and/or the quaternized amine according to the general formula (Tkat3) is an amido amine and/or a quaternized amido amine in which R1 denotes a branched or unbranched, saturated or unsaturated acyl residue having 6 to 30 C atoms, which can include at least one OH group, is preferred. A fatty acid residue from oils and waxes, in particular from natural oils and waxes, is preferred here. Suitable examples include lanolin, beeswax or candelilla wax.

Also preferred are amido amines and/or quaternized amido amines in which R2, R3 and/or R4 in formula (Tkat3) denote a residue according to the general formula CH₂CH₂OR5, in which R5 can have the meaning of alkyl residues having 1 to 4 carbon atoms, hydroxyethyl or hydrogen. The preferred value of n in the general formula (Tkat8) is an integer between 2 and 5.

The alkyl amido amines can be present as is and can also be converted into a quaternary compound in the composition by protonation in a correspondingly acid solution. Cationic alkyl amido amines are preferred according to the invention.

Examples of such commercial products according to the invention are Witcamine® 100, Incromine® BB, Mackine® 401 and other Mackine® types, Adogen® S18V, and as permanently cationic amino amines: Rewoquat® RTM 50, Empigen® CSC, Swanol® Lanoquat DES-50, Rewoquat® UTM 50, Schercoquat® BAS, Lexquat® AMG-BEO, or Incroquat® Behenyl HE.

The aforementioned cationic surfactants can be used individually or in any combinations with one another, wherein they are include d in amounts of between 0.01 and 10 wt. %, preferably in amounts from 0.01 to 7.5 wt. % and most particularly preferably in amounts from 0.1 to 5.0 wt. %. The very best results are obtained with amounts from 0.1 to 3.0 wt. %, relative in each case to the total composition of the individual agent.

The cationic and/or amphoteric polymers can be homopolymers or copolymers or polymers based on natural polymers, wherein the quaternary nitrogen groups can be include d either in the polymer chain or preferably as a substituent on one or more of the monomers. The ammonium group-including monomers can be copolymerized with non-cationic monomers. Suitable cationic monomers are unsaturated, radically polymerizable compounds bearing at least one cationic group, in particular ammonium-substituted vinyl monomers such as for example trialkyl methacryloxy alkylammonium, trialkyl acryloxy alkylammonium, dialkyl diallyl ammonium and quaternary vinyl ammonium monomers with cyclic groups including cationic nitrogens, such as pyridinium, imidazolium or quaternary pyrrolidones, e.g. alkyl vinylimidazolium, alkyl vinylpyridinium, or alkyl vinylpyrrolidone salts. The alkyl groups of these monomers are preferably low alkyl groups such as for example C1 to C7 alkyl groups, particularly preferably C1 to C3 alkyl groups.

The ammonium group-including monomers can be copolymerized with non-cationic monomers. Suitable comonomers are for example acrylamide, methacrylamide, alkyl and dialkyl acrylamide, alkyl and dialkyl methacrylamide, alkyl acrylate, alkyl methacrylate, vinyl caprolactone, vinylcaprolactam, vinylpyrrolidone, vinyl esters, for example vinyl acetate, vinyl alcohol, propylene glycol or ethylene glycol, the alkyl groups of these monomers preferably being C1 to C7 alkyl groups, particularly preferably C1 to C3 alkyl groups.

A highly preferred polymer is commercially available under the name Polyquaternium-74.

A further preferred cationic polymer comprises at least one structural unit of formula (I), at least one structural unit of formula (II), at least one structural unit of formula (III) and at least one structural unit of formula (IV),

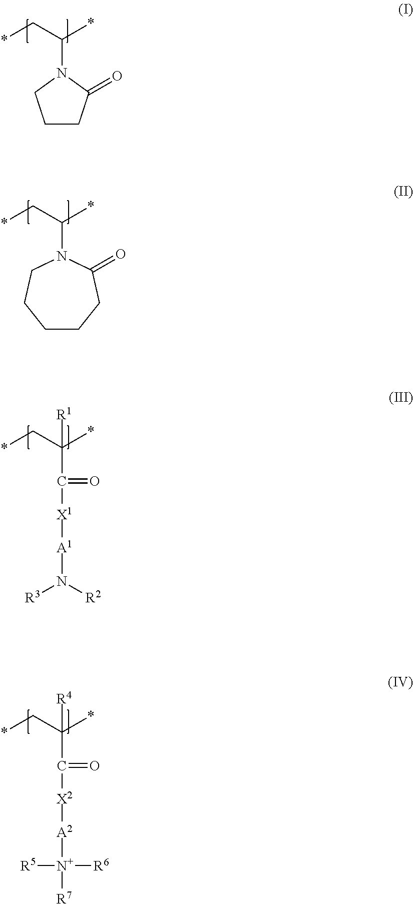

in which
R¹ and R⁴ independently of one another denote a hydrogen atom or a methyl group,
X¹ and X² independently of one another denote an oxygen atom or an NH group,
A¹ and A² independently of one another denote an ethane-1,2-diyl, propane-1,3-diyl or butane-1,4-diyl group,
R², R³, R⁵ and R⁶ independently of one another denote a (C₁ to C₄) alkyl group,
R⁷ denotes a (C₈ to C₃₀) alkyl group

In the above formulae and all subsequent formulae, a chemical bond marked with the symbol * denotes a free valence of the corresponding structural fragment. All possible physiologically acceptable anions, such as for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge in the agent according to the invention.

Examples of (C₁ to C₄) alkyl groups according to the invention are methyl, ethyl, propyl, isopropyl, butyl, sec-butyl, isobutyl, tert-butyl.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl).

The cationic polymers according to the invention preferably have a molecular weight from 10,000 g/mol to 50,000,000 g/mol, in particular from 50,000 g/mol to 5,000,000 g/mol, particularly preferably from 75,000 g/mol to 1,000,000 g/mol.

Preferred agents within the meaning of the invention include these aforementioned cationic polymers in an amount from 0.1 wt. % to 20.0 wt. % particularly preferably from 0.2 wt. % to 10.0 wt. %, most particularly preferably from 0.5 wt. % to 5.0 wt. %, relative in each case to the weight of the agent.

According to the invention the following cationic polymers are highly preferably used in the agents according to the invention if the cationic polymers satisfy one or more of the following features in regard to the aforementioned formulae (I) to (IV):
R¹ and R⁴ each denote a methyl group,

X¹ denotes an NH group,

X² denotes an NH group,

A¹ and A² independently of one another denote ethane-1,2-diyl or propane-1,3-diyl,
R², R³, R⁵ and R⁶ independently of one another denote methyl or ethyl (particularly preferably methyl),
R⁷ denotes a (C₁₀ to C₂₄) alkyl group, in particular decyl (caprinyl), dodecyl (lauryl), tetradecyl (myristyl), hexadecyl (cetyl), octadecyl (stearyl), eicosyl (arachyl) or docosyl (behenyl).

It is preferable according to the invention to select the structural unit of formula (III) from at least one structural unit of formula (III-1) to (III-8)

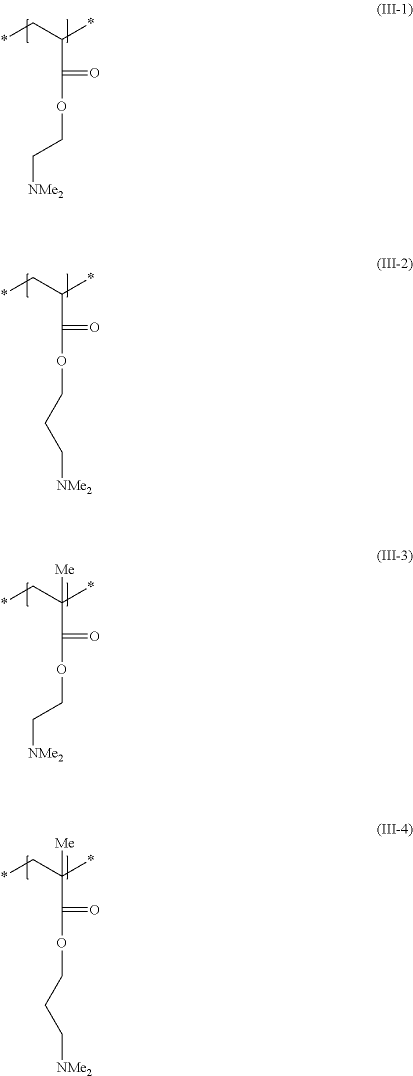

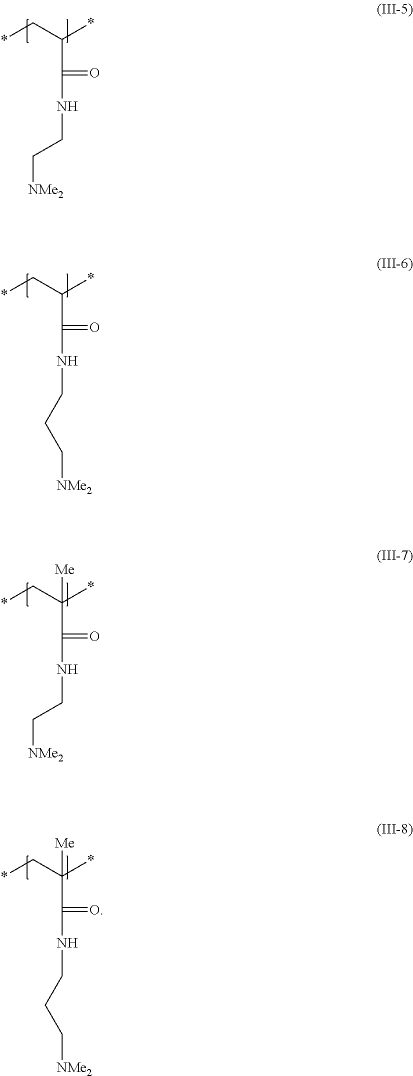

It has moreover proved particularly preferable to choose the structural unit according to formula (III-7) and/or formula (III-8) as the structural unit of formula (III). According to the invention the structural unit of formula (III-8) is a most particularly preferred structural unit.

With a view to achieving the object it has furthermore proved preferable for the structural unit of formula (IV) to be selected from at least one structural unit of formulae (IV-1) to (IV-8)

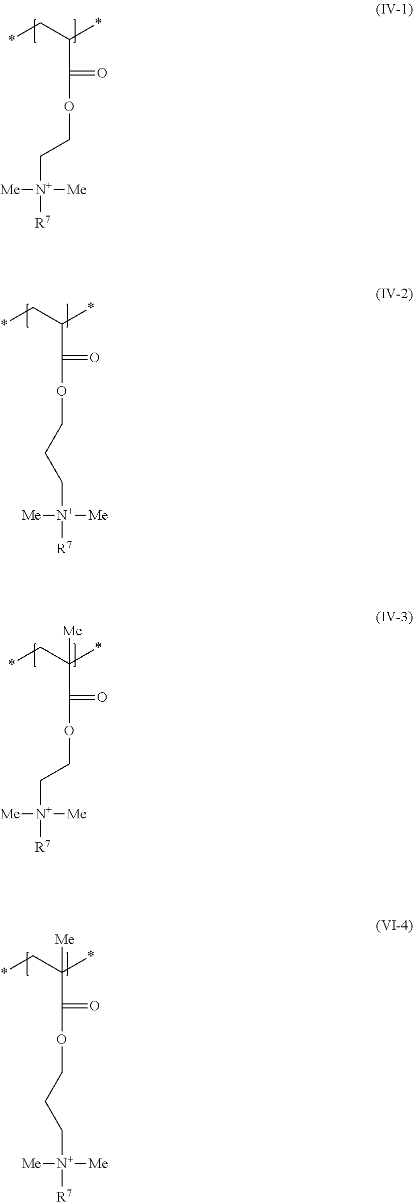

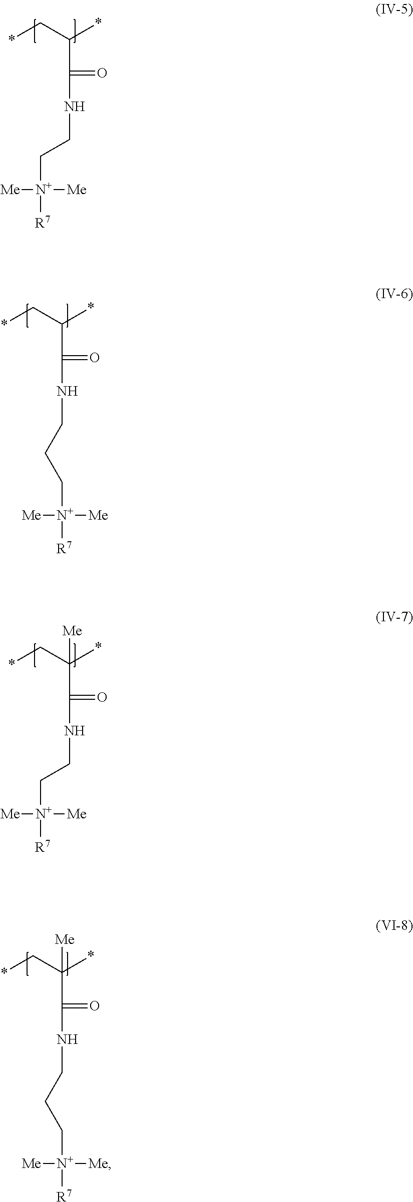

in which R⁷ in each case denotes a (C₈ to C₃₀) alkyl group.

The structural units of formula (IV-7) and/or formula (IV-8), in which in each case R⁷ denotes octyl (capryl), decyl (caprinyl), dodecyl (lauryl), tetradecyl (myristyl), hexadecyl (cetyl), octadecyl (stearyl), eicosyl (arachyl) or docosyl (behenyl), are in turn regarded as a particularly preferred structural unit of formula (IV). According to the invention the structural unit of formula (IV-8) is a most particularly preferred structural unit of formula (IV).

A cationic polymer that is most particularly preferably include d in the agent according to the invention comprises at least one structural unit of formula (I), at least one structural unit of formula (II), at least one structural unit of formula (III-8) and at least one structural unit of formula (IV-8),

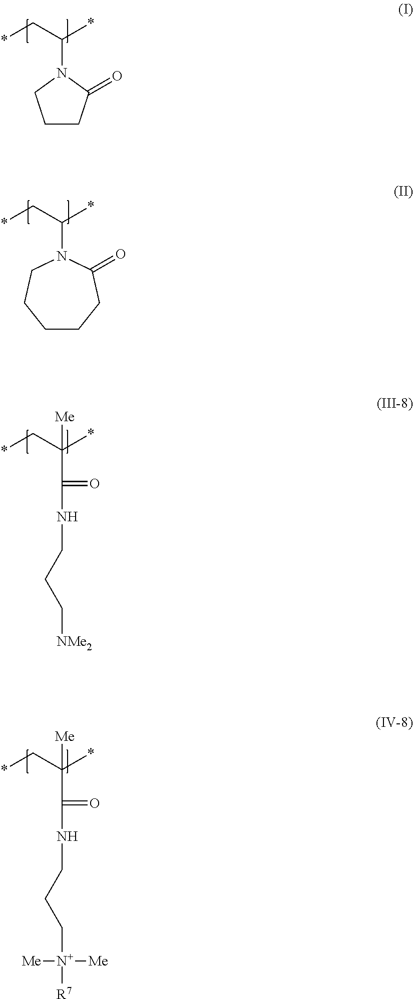

in which R⁷ denotes octyl (capryl), decyl (caprinyl), dodecyl (lauryl), tetradecyl (myristyl), hexadecyl (cetyl), octadecyl (stearyl), eicosyl (arachyl) or docosyl (behenyl).

A most particularly preferred cationic polymer according to the invention is the copolymer of N-vinylpyrrolidone, N-vinylcaprolactam, N-(3-dimethylaminopropyl) methacrylamide and 3-(methacryloylamino)propyl lauryl dimethylammonium chloride (INCI name: Polyquaternium-69), which is sold for example by ISP under the trade name AquaStyle® 300 (28-32 wt. % active substance in an ethanol-water blend, molecular weight 350,000).

A particularly suitable homopolymer is the optionally crosslinked poly(methacryloyloxyethyl trimethylammonium chloride) with the INCI name Polyquaternium-37. Such products are commercially available for example under the names Rheocare® CTH (Cosmetic Rheologies) and Synthalen® CR (3V Sigma).

The homopolymer is preferably used in the form of a non-aqueous polymer dispersion. Such polymer dispersions are commercially available under the names Salcare® SC 95 and Salcare® SC 96.

Suitable cationic polymers that are derived from natural polymers are cationic derivatives of polysaccharides, for example cationic derivatives of cellulose, starch or guar. Also suitable are chitosan and chitosan derivatives. Cationic polysaccharides have the general formula G-O—B—N⁺R^{a}R_{b}R_{c}A⁻
G is an anhydroglucose residue, for example starch or cellulose anhydroglucose;
B is a divalent group of compounds, for example alkylene, oxyalkylene, polyoxyalkylene or hydroxyalkylene;
Rₐ, R_{b} and R_{c} are independently of one another alkyl, aryl, alkylaryl, arylalkyl, alkoxyalkyl or alkoxyaryl, each having up to 18 C atoms, the total number of C atoms in Rₐ, R_{b} and R_{c} preferably being a maximum of 20;
A⁻ is a conventional counteranion and is preferably chloride.

Cationic, i.e. quaternized, celluloses are available on the market with varying degrees of substitution, cationic charge density, nitrogen content and molecular weights. For example, Polyquaternium-67 is commercially available under the names Polymer® SL or Polymer® SK (Amerchol). A further highly preferred cellulose is available under the trade name Mirustyle® CP from Croda. This is a trimonium and cocodimonium hydroxyethylcellulose as a derivatized cellulose with the INCI name Polyquaternium-72. Polyquaternium-72 can be used both in solid form and pre-dissolved in aqueous solution.

Further cationic celluloses are available under the names Polymer JR® 400 (Amerchol, INCI name Polyquatemium-10) and Polymer Quatrisoft® LM-200 (Amerchol, INCI name Polyquaternium-24). Further commercial products are the compounds Celquat® H 100 and Celquat® L 200, Particularly preferred cationic celluloses are Polyquatemium-24, Polyquaternium-67 and Polyquaternium-72.

Suitable cationic guar derivatives are sold under the trade name Jaguar and have the INCI name Guar Hydroxypropyltrimonium Chloride. Particularly suitable cationic guar derivatives are also sold furthermore by Hercules under the name N-Hance®. Further cationic guar derivatives are sold by Cognis under the name Cosmedia®. A preferred cationic guar derivative is the commercial product AquaCat® from Hercules. This raw material is a pre-dissolved cationic guar derivative. The cationic guar derivatives are preferred according to the invention.

A suitable chitosan is sold for example by Kyowa Oil & Fat, Japan, under the trade name Flonac®. A preferred chitosan salt is chitosonium pyrrolidone carboxylate, which is sold for example under the name Kytamer® PC by Amerchol, USA. Further chitosan derivatives are freely available commercially under the trade names Hydagen® CMF, Hydagen® HCMF and Chitolam® NB/101.

Further preferred cationic polymers are for example
cationic alkyl polyglycosides,
cationized honey, for example the commercial product Honeyquat® 50,
polymeric dimethyldiallyl ammonium salts and copolymers thereof with esters and amides of acrylic acid and methacrylic acid. The products available commercially under the names Merquat® 100 (poly(dimethyldiallyl ammonium chloride)) and Merquat® 550 (dimethyldiallyl ammonium chloride-acrylamide copolymer) are examples of such cationic polymers with the INCI name Polyquaternium-7,
vinylpyrrolidone-vinylimidazolium methochloride copolymers, such as are sold under the names Luviquat® FC 370, FC 550 and the INCI name Polyquatemium-16 as well as FC 905 and HM 552,
quaternized vinylpyrrolidone/dimethylaminoethyl methacrylate, for example vinylpyrrolidone/dimethylaminoethyl methacrylate methosulfate copolymer, which is sold under the trade names Gafquat® 755 N and Gafquat® 734 by Gaf Co., USA and the INCI name Polyquatemium-11,
quaternized polyvinyl alcohol,
and the polymers known under the names Polyquaternium-2, Polyquaternium-17, Polyquaternium-18 and Polyquaternium-27 with quaternary nitrogen atoms in the polymer main chain,
vinylpyrrolidone-vinylcaprolactam-acrylate terpolymers, such as are available commercially with acrylic acid esters and acrylic acid amides as the third monomer unit under the name Aquaflex® SF 40, for example.

Particularly preferred amphoteric polymers are copolymers of at least one monomer (Mono1) or (Mono2) with the monomer (Mono3), in particular copolymers of monomers (Mono2) and (Mono3). Amphoteric polymers that are most particularly preferably used according to the invention are copolymers of diallyldimethylammonium chloride and acrylic acid. These copolymers are sold under the INCI name Polyquaternium-22, inter alia under the trade name Merquat® 280 (Nalco).

Amphoteric polymers based on a comonomer (Mono4) that are most particularly preferably used according to the invention are terpolymers of diallyldimethylammonium chloride, acrylamide and acrylic acid. These copolymers are sold under the INCI name Polyquatemium-39, inter alia under the trade name Merquat® Plus 3330 (Nalco).

The amphoteric polymers can generally be used according to the invention both directly and in the form of the salt, which is obtained by neutralization of the polymers, with an alkali hydroxide for example.

The polymers described thus far represent only some of the polymers that can be used according to the invention. To avoid having to list and describe the composition of all cationic and/or amphoteric polymers that are suitable according to the invention, the INCI names of the polymers that are preferred according to the invention are given in summary. The polymers that are preferred according to the invention have the INCI names: Polyquatemium-2, Polyquatemium-4, Polyquatemium-6, Polyquaternium-7, Polyquatemium-10, Polyquaternium-11, Polyquatemium-15, Polyquatemium-16, Polyquaternium-17, Polyquaternium-18, Polyquatemium-22, Polyquatemium-24, Polyquatemium-28, Polyquaternium-32, Polyquatemium-33, Polyquatemium-34, Polyquatemium-35, Polyquatemium-37, Polyquatemium-39, Polyquatemium-41, Polyquaternium-42, Polyquaternium-44, Polyquatemium-47, Polyquaternium-55, Polyquaternium-67, Polyquaternium-68, Polyquaternium-69, Polyquaternium-72, Polyquaternium-74, Polyquaternium-76, Polyquatemium-86, Polyquaternium-89 and Polyquaternium-95 as well as mixtures thereof.

The aforementioned cationic polymers can be used individually or in any combinations with one another, wherein they are include d in amounts of between 0.01 and 10 wt. %, preferably in amounts from 0.01 to 7.5 wt. % and most particularly preferably in amounts from 0.1 to 5.0 wt. %. The very best results are obtained with amounts from 0.1 to 3.0 wt. %, relative in each case to the total composition of the individual agent.

Furthermore, all ingredients that are conventionally used in cosmetic compositions can be added to this highly preferred framework of ingredients.

In addition to the aforementioned required silicones, the compositions according to the invention can include further silicones. These optional silicones are preferably at least one silicone polymer selected from the group of dimethiconols and/or the group of amino-functional silicones and/or the group of dimethicones and/or the group of cyclomethicones.

The dimethicones according to the invention can be both linear and branched and also cyclic or cyclic and branched. Linear dimethicones can be represented by the following structural formula (Si1):

(SiR¹₃)—O—(SiR²₂—O—)ₓ—(SiR¹₃) (Si1)

Branched dimethicones can be represented by the structural formula (Si1.1):

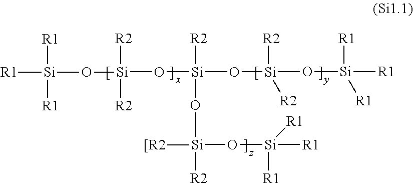

Residues R¹ and R² independently of one another each denote hydrogen, a methyl residue, a C2 to C30 linear, saturated or unsaturated hydrocarbon residue, a phenyl residue and/or an aryl residue. The numbers x, y and z are integers and each run independently of one another from 0 to 50,000. The molecular weights of the dimethicones are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs. Viscosities around the range of approximately 60,000 cPs are highly preferred. Reference is made here by way of example to the product “Dow Corning 200 with 60,000 cSt”.

Particularly preferred cosmetic or dermatological preparations according to the invention are characterized in that they include at least one silicone of formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are include d in the compositions according to the invention in amounts from 0.01 to 10 wt. %, preferably 0.01 to 8 wt. %, particularly preferably 0.1 to 7.5 wt. % and in particular 0.1 to 5 wt. %, relative to the total composition.

Finally, the silicone compounds are understood to include dimethiconols (Si8). The dimethiconols according to the invention can be both linear and branched and also cyclic or cyclic and branched. Linear dimethiconols can be represented by the following structural formula (Si8-I):

(SiOHR¹₂)—O—(SiR²₂—O—)ₓ—(SiOHR¹₂) (Si8-1)

Branched dimethiconols can be represented by the structural formula (Si8-II):

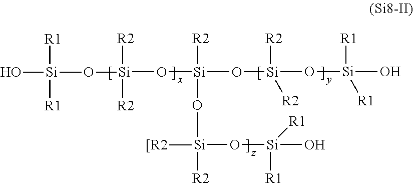

Residues R¹ and R² independently of one another each denote hydrogen, a methyl residue, a C2 to C30 linear, saturated or unsaturated hydrocarbon residue, a phenyl residue and/or an aryl residue. The numbers x, y and z are integers and each run independently of one another from 0 to 50,000. The molecular weights of the dimethiconols are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs.

The following commercial products are cited as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (these last four all GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (these last all Wacker-Chemie GmbH). The dimethiconols (Si8) are include d in the compositions according to the invention in amounts from 0.01 to 10 wt. %, preferably 0.01 to 8 wt. %, particularly preferably 0.1 to 7.5 wt. % and in particular 0.1 to 5 wt. % of dimethiconol, relative to the composition. Particularly preferred agents according to the invention include one or more amino-functional silicones. Such silicones can be described for example by the formula (Si-2)

M(RₐQ_{b}SiO_{(4-a-b)/2})ₓ(R_{c}SiO_{(4-c)/2})_{y}M (Si-2)

in which in the above formula
R is a hydrocarbon or a hydrocarbon residue having 1 to approximately 6 carbon atoms,
Q is a polar residue of the general formula —R¹HZ, in which
   R¹ is a divalent linking group that, is bound to hydrogen and to the residue Z, composed of carbon and hydrogen atoms, carbon, hydrogen and oxygen atoms or carbon, hydrogen and nitrogen atoms, and
   Z is an organic, amino-functional residue including at least one amino-functional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2000, preferably from approximately 3 to approximately 50 and most preferably from approximately 3 to approximately 25, and
y is a number in the range from approximately 20 to approximately 10,000, preferably from approximately 125 to approximately 10,000 and most preferably from approximately 150 to approximately 1000, and
M is a suitable silicone end group as is known in the prior art, preferably trimethylsiloxy.

According to formula (Si-2) Z is an organic, amino-functional residue including at least one functional amino group. A possible formula for said Z is NH(CH₂)_{z}NH₂, in which z is an integer greater than or equal to 1. Another possible formula for said Z is —NH(CH₂)_{z}(CH₂)_{zz}NH, in which both z and zz are independently of one another an integer greater than or equal to 1, this structure comprising diamino ring structures, such as piperazinyl. Said Z is most preferably an —NHCH₂CH₂NH₂ residue. Another possible formula for said Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0. Q according to formula (Si-2) is most preferably a polar amino-functional residue of the formula —CH₂CH₂CH₂NHCH₂CH₂NH₂.

In formula (Si-2) a assumes values in the range from 0 to 2, b assumes values in the range from 2 to 3, a+b is less than or equal to 3, and c is a number in the range from 1 to 3.

Cationic silicone oils such as for example the commercially available products Dow Corning (DC) 929 Emulsion, DC 2-2078, DC 5-7113, SM-2059 (General Electric) and SLM-55067 (Wacker) are suitable according to the invention.

Particularly preferred agents according to the invention are characterized in that they include at least one amino-functional silicone of formula (Si-3a)

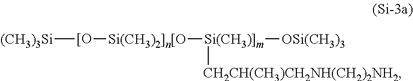

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10. Under the INCI declaration these silicones are known as trimethylsilylamodimethicones and they are available for example under the name Q2-7224 (manufacturer: Dow Corning; a stabilized trimethylsilylamodimethicone).

Also particularly preferred are agents according to the invention that include an amino-functional silicone of formula (Si-3b)

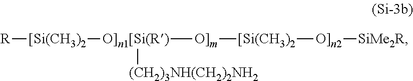

in which
R denotes —OH, an (optionally ethoxylated and/or propoxylated) (C₁ to C₂₀) alkoxy group or a —CH₃ group,
R′ denotes —OH, a (C₁ to C₂₀) alkoxy group or a —CH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Under the INCI declaration these silicones are known as amodimethicones or as functionalized amodimethicones, such as for example Bis(C13-15 Alkoxy) PG Amodimethicone (available for example as the commercial product DC 8500 from Dow Corning), Trideceth-9 PG-Amodimethicone (available for example as the commercial product Silcare Silicone SEA from Clariant).

Suitable diquaternary silicones are selected from compounds of the general formula (Si3c)

[R¹R²R³N⁺-A-SiR⁷R⁸—)(O—SiR⁹R¹⁰)ₙ—O—SiR¹¹R¹²-A-N⁺R⁴R⁵R⁶]2X⁻ (Si3c)

in which residues R1 to R6 independently of one another denote C1 to C22 alkyl residues, which can include hydroxyl groups, and wherein preferably at least one of the residues has at least 8 C atoms and the other residues have 1 to 4 C atoms,
residues R⁷ to R¹² independently of one another are identical or different and denote C1 to C10 alkyl or phenyl, A denotes a divalent organic group of compounds,
n is a number from 0 to 200, preferably from 10 to 120, particularly preferably from 10 to 40,
and X⁻ is an anion.

The divalent group of compounds is preferably a C1 to C12 alkylene or alkoxyalkylene group, which can be substituted with one or more hydroxyl groups. The group —(CH₂)₃—O—CH₂—CH(OH)—CH₂— is particularly preferred.

The anion X⁻ can be a halide ion, an acetate, an organic carboxylate or a compound of the general formula RSO₃⁻, in which R has the meaning of C1 to C4 alkyl residues.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

in which A is the group —(CH₂)₃—O—CH₂—CH(OH)—CH₂—,
R is an alkyl residue having at least 8 C atoms and n is a number from 10 to 120.

Suitable silicone polymers having two terminal, quaternary ammonium groups are known under the INCI name Quaternium-80. These are dimethyl siloxanes having two terminal trialkylammonium groups. Such diquaternary polydimethyl siloxanes are sold by Evonik under the trade names Abil® Quat 3270, 3272 and 3474.

Hair treatment agents that are preferred according to the invention are characterized in that they contain, relative to their weight, 0.01 to 10 wt. %, preferably 0.01 to 8 wt. %, particularly preferably 0.1 to 7.5 wt. % and in particular 0.2 to 5 wt. % of amino-functional silicone(s) and/or diquaternary silicone.

Polyammonium-polysiloxane compounds represent a further silicone according to the invention having amino functions. The polyammonium-polysiloxane compounds can be purchased for example from GE Bayer Silicones under the trade name Baysilone®. The products with the names Baysilone TP 3911, SME 253 and SFE 839 are preferred here. The use of Baysilone TP 3911 as the active component of the compositions according to the invention is most particularly preferred. The polyammonium-polysiloxane compounds are used in the compositions according to the invention in an amount from 0.01 to 10 wt. %, preferably 0.01 to 7.5, particularly preferably 0.01 to 5.0 wt. %, most particularly preferably from 0.05 to 2.5 wt. %, relative in each case to the total composition.

The cyclic dimethicones referred to under INCI as cyclomethicones can also be used to advantage according to the invention. Cosmetic or dermatological preparations according to the invention are preferred that include at least one silicone of formula (Si-4),

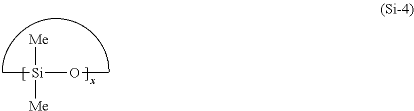

in which x denotes a number from 3 to 200, preferably from 3 to 10, more preferably from 3 to 7 and in particular 3, 4, 5 or 6.

Agents that are likewise preferred according to the invention are characterized in that they include at least one silicone of formula (Si-5)

R₃Si—[O—SiR₂]ₓ—(CH₂)ₙ—[O—SiR₂]_{y}—O—SiR₃ (Si-5),

in which R denotes identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As further silicones in addition to the dimethicones, dimethiconols, amodimethicones and/or cyclomethicones according to the invention, water-soluble silicones can be include d in the compositions according to the invention.

Corresponding hydrophilic silicones are selected for example from the compounds of formulae (Si-6) and/or (Si-7). Silicone-based water-soluble surfactants that are preferred in particular are selected from the group of dimethicone copolyols, which are preferably alkoxylated, in particular polyethoxylated or polypropoxylated.

According to the invention dimethicone copolyols are understood to be preferably polyoxyalkylene-modified dimethyl polysiloxanes of the general formulae (Si-6) or (Si-7):

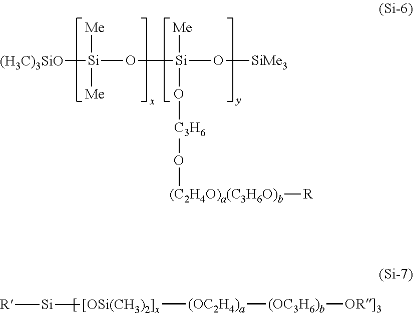

in which the residue R denotes a hydrogen atom, an alkyl group having 1 to 12 C atoms, an alkoxy group having 1 to 12 C atoms or a hydroxyl group, the residues R′ and R″ denote alkyl groups having 1 to 12 C atoms, x denotes an integer from 1 to 100, preferably from 20 to 30, y denotes an integer from 1 to 20, preferably from 2 to 10, and a and b denote integers from 0 to 50, preferably from 10 to 30.

Particularly preferred dimethicone copolyols within the meaning of the invention are for example the products sold commercially under the trade name SILWET (Union Carbide Corporation) and DOW CORNING. Dimethicone copolyols that are particularly preferred according to the invention are Dow Corning 190 and Dow Corning 193.

The dimethicone copolyols are include d in the compositions according to the invention in amounts from 0.01 to 10 wt. %, preferably 0.01 to 8 wt. %, particularly preferably 0.1 to 7.5 wt. % and in particular 0.1 to 5 wt. % of dimethicone copolyol, relative to the composition.

Ester oils can be include d to particular advantage as oil bodies in active ingredient combinations according to the invention. Ester oils are defined as follows:

Ester oils are understood to be the esters of C₆-C₃₀ fatty acids with C2-C30 fatty alcohols. The monoesters of fatty acids with alcohols having 2 to 24 C atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

The ester oils can naturally also be alkoxylated with ethylene oxide, propylene oxide or mixtures of ethylene oxide and propylene oxide. The alkoxylation can take place both on the fatty alcohol part and on the fatty acid part and also on both parts of the ester oils. It is preferred according to the invention, however, for the fatty alcohol to be alkoxylated first and then esterified with fatty acid. These compounds are shown in general in formula (D4-II).

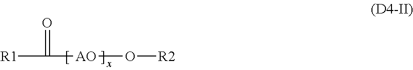

R1 here denotes a saturated or unsaturated, branched or unbranched, cyclic saturated, cyclic unsaturated acyl residue having 6 to 30 carbon atoms,
AO denotes ethylene oxide, propylene oxide or butylene oxide,
X denotes a number between 1 and 200, preferably between 1 and 100, particularly preferably between 1 and 50, most particularly preferably between 1 and 20, highly preferably between 1 and 10 and most preferably between 1 and 5,
R2 denotes a saturated or unsaturated, branched or unbranched, cyclic saturated, cyclic unsaturated alkyl, alkenyl, alkynyl, phenyl or benzyl residue having 6 to 30 carbon atoms.

Examples of fatty acid components used as residue R1 in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components as residue R2 in the ester oils are benzyl alcohol, isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. An ester oil that is particularly preferred according to the invention is available for example under the INCI name PPG-3 Benzyl Ether Myristate.

Ester oils are also understood to include:
dicarboxylic acid esters such as di-n-butyl adipate, di-(2-ethylhexyl) adipate, di-(2-ethylhexyl)succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate, and
symmetrical, asymmetrical or cyclic esters of carbonic acid with fatty alcohols, for example glycerol carbonate or dicaprylyl carbonate (Cetiol® CC),
tri-fatty acid esters of saturated and/or unsaturated linear and/or branched fatty acids with glycerol,
fatty acid partial glycerides, namely monoglycerides, diglycerides and technical mixtures thereof. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

The ester oils are used in the agents according to the invention in an amount from 0.01 to 20 wt. %, preferably 0.01 to 10.0 wt. %, particularly preferably 0.01 to 7.5 wt. %, highly preferably from 0.1 to 5.0 wt. %. Naturally it is also possible according to the invention to use a plurality of ester oils at the same time.

Further oil bodies according to the invention are:
vegetable oils. Examples of such oils are sunflower oil, olive oil, soybean oil, rapeseed oil, almond oil, jojoba oil, orange oil, wheat germ oil, peach kernel oil and the liquid components of coconut oil. Other triglyceride oils are also suitable, however, such as the liquid components of beef fat and synthetic triglyceride oils
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds available as commercial products, 1,3-di-(2-ethylhexyl)cyclohexane (Cetiol® S) and di-n-octyl ether (Cetiol® OE), can be preferred.

Amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, corn oil, almond oil, manila oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil, for example, are used as natural oils.

In addition to the active ingredient combination according to the invention the hair treatment agents according to the invention naturally also include further constituents that are conventionally used in cosmetic compositions. The choice of these constituents is generally governed by the intended use of the hair treatment agents. In the case of a shampoo, for example, further surface-active substances are include d. In the case of hair masks, further cationic compounds and further care substances are optionally include d. In many cases the agents include at least one surface-active substance, with both anionic and zwitterionic, ampholytic, non-ionic and cationic surface-active substances being suitable in principle. The choice of surface-active substances is governed by the type of agent.

All anionic surface-active substances that are suitable for use on the human body are suitable as anionic surfactants (Tanion) in preparations according to the invention. Typical examples of anionic surfactants are:
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16, and salts thereof,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
hydroxyl sulfonates substantially corresponding to at least one of the following two formulae or to mixtures and salts thereof,

CH₃—(CH₂)_{y}—CHOH—(CH₂)ₚ—(CH—SO₃M)-(CH₂)_{z}—CH₂—O—(CₙH₂ₙO)ₓ—H, and/or

CH₃—(CH₂)_{y}—(CH—SO₃M)-(CH₂)ₚ—CHOH—(CH₂), —CH₂—O—(CₙH₂ₙO)ₓ—H
wherein in both formulae y and z=0 or integers from 1 to 18, p=0, 1 or 2 and the sum (y+z+p) is a number from 12 to 18, x=0 or a number from 1 to 30 and n is an integer from 2 to 4 and M=H or an alkali ion, in particular sodium, potassium, lithium, alkaline-earth ion, in particular magnesium, calcium, zinc and/or an ammonium ion, which can optionally be substituted, in particular mono-, di-, tri- or tetraammonium ions with C1 to C4 alkyl, alkenyl or aryl residues,
sulfated hydroxyalkyl polyethylene and/or hydroxyalkylene propylene glycol ethers of the formula R¹—(CHOSO₃M)-CHR³—(OCHR⁴—CH₂)ₙ—OR² in which R¹ denotes a linear alkyl residue having 1 to 24 C atoms, R² denotes a linear or branched, saturated alkyl residue having 1 to 24 C atoms, R³ denotes hydrogen or a linear alkyl residue having 1 to 24 C atoms, R⁴ denotes hydrogen or a methyl residue and M denotes hydrogen, ammonium, alkyl ammonium, alkanol ammonium, wherein the alkyl and alkanol residues each have 1 to 4 C atoms, or a metal atom selected from lithium, sodium, potassium, calcium or magnesium, and n denotes a number in the range from 0 to 12 and furthermore the total number of C atoms include d in R¹ and R³ is 2 to 44,
sulfonates of unsaturated fatty acids having 8 to 24 C atoms and 1 to 6 double bonds,
esters of tartaric acid and citric acid with alcohols that are addition products of approximately 2 to 15 molecules of ethylene oxide and/or propylene oxide with fatty alcohols having 8 to 22 C atoms,
alkyl and/or alkenyl ether phosphates of the formula

R¹(OCH₂CH₂)ₙ—O—(PO—OX)—OR²,

in which R¹ preferably denotes an aliphatic hydrocarbon residue having 8 to 30 carbon atoms, R² denotes hydrogen, a (CH₂CH₂O)ₙR² residue or X, n denotes numbers from 1 to 10 and X denotes hydrogen, an alkali or alkaline-earth metal or NR³R⁴R⁵R⁶, with R³ to R⁶ independently of one another denoting hydrogen or a C1 to C4 hydrocarbon residue,
sulfated fatty acid alkylene glycol esters of the formula RCO(AlkO)ₙSO₃M in which RCO— denotes a linear or branched, aliphatic, saturated and/or unsaturated acyl residue having 6 to 22 C atoms, Alk denotes CH₂CH₂, CHCH₃CH₂ and/or CH₂CHCH₃, n denotes numbers from 0.5 to 5 and M denotes a metal, such as alkali metal, in particular sodium, potassium, lithium, alkaline-earth metal, in particular magnesium, calcium, zinc, or ammonium ion, such as ⁺NR³R⁴R⁵R⁶, with R³ to R⁶ independently of one another denoting hydrogen or a C1 to C4 hydrocarbon residue, monoglyceride sulfates and monoglyceride ether sulfates of the formula

R⁸OC—(OCH₂CH₂)ₓ—OCH₂—[CHO(CH₂CH₂O)_{y}H]—CH₂—O—(CH₂CH₂O)_{z}—SO₃X,
in which R⁸CO denotes a linear or branched acyl residue having 6 to 22 carbon atoms, x, y and z in total denote 0 or numbers from 1 to 30, preferably 2 to 10, and X denotes an alkali or alkaline-earth metal. Typical examples of suitable monoglyceride (ether) sulfates within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as the ethylene oxide adducts thereof with sulfur trioxide or chlorosulfonic acid in the form of their sodium salts. Monoglyceride sulfates are preferably used in which R⁸CO denotes a linear acyl residue having 8 to 18 carbon atoms,
amide ether carboxylic acids, R¹—CO—NR²—CH₂CH₂—O—(CH₂CH₂O)ₙCH₂COOM, with R¹ as a straight-chain or branched alkyl or alkenyl residue having a number of carbon atoms in the chain from 2 to 30, n denotes an integer from 1 to 20 and R² denotes hydrogen, a methyl, ethyl, propyl, isopropyl, n-butyl, t-butyl or isobutyl residue and M denotes hydrogen or a metal such as alkali metal, in particular sodium, potassium, lithium, alkaline-earth metal, in particular magnesium, calcium, zinc, or an ammonium ion, such as ⁺NR³R⁴R⁵R⁶, with R³ to R⁶ independently of one another denoting hydrogen or a C₁ to C₄ hydrocarbon residue. Such products are available for example from Chem-Y under the product name Akypo®,
acyl glutamates of the formula XOOC—CH₂CH₂CH(C(NH)OR)—COOX, in which RCO denotes a linear or branched acyl residue having 6 to 22 carbon atoms and 0 and/or 1, 2 or 3 double bonds and X denotes hydrogen, an alkali and/or alkaline-earth metal, ammonium, alkyl ammonium, alkanol ammonium or glucammonium,
condensation products of a water-soluble salt of a water-soluble protein hydrolysate with a C8-C30 fatty acid. Such products have long been available commercially under the trademark Lamepon®, Maypon®, Gluadin®, Hostapon® KCG or Amisoft®,
alkyl and/or alkenyl oligoglycoside carboxylates, sulfates, phosphates and/or isethionates,
acyl lactylates and
hydroxy mixed ether sulfates.

If the gentle anionic surfactants include polyglycol ether chains, it is most particularly preferable for them to have a narrow homolog distribution. In the case of gentle anionic surfactants having polygycol ether units, it is furthermore preferable for the number of glycol ether groups to be 1 to 20, preferably 2 to 15, particularly preferably 2 to 12. Particularly gentle anionic surfactants having polyglycol ether groups without a narrow homolog distribution can also be obtained for example if the number of polyglycol ether groups is 4 to 12 and Zn or Mg ions are chosen as the counterion. One example is the commercial product Texapon® ASV.

Non-ionic surfactants (Tnio) are for example
addition products of 2 to 50 mol of ethylene oxide and/or 0 to 5 mol of propylene oxide with linear and branched fatty alcohols having 6 to 30 C atoms, fatty alcohol polyglycol ethers or fatty alcohol polypropylene glycol ethers or mixed fatty alcohol polyethers,
addition products of 2 to 50 mol of ethylene oxide and/or 0 to 5 mol of propylene oxide with linear and branched fatty acids having 6 to 30 C atoms, fatty acid polyglycol ethers or fatty acid polypropylene glycol ethers or mixed fatty acid polyethers,
addition products of 2 to 50 mol of ethylene oxide and/or 0 to 5 mol of propylene oxide with linear and branched alkylphenols having 8 to 15 C atoms in the alkyl group, alkylphenol polyglycol ethers or alkyl polypropylene glycol ethers or mixed alkylphenol polyethers,
addition products of 2 to 50 mol of ethylene oxide and/or 0 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C2 to C6 alkyl residue, such as for example the types available under the commercial names Dehydrol® LS, Dehydrol® LT (Cognis),
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as for example the commercial product Hydagen® HSP (Cognis) or Sovermol® types (Cognis),
alkoxylated triglycerides,
alkoxylated fatty acid alkyl esters of the formula (Tnio-1)

R¹CO—(OCH₂CHR²)_{w}OR³ (Tnio-1)
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,
amine oxides,
hydroxy mixed ethers, R¹O[CH₂CH(CH₃)O]ₓ(CH₂CHR²O)_{y}[CH₂CH(OH)R³]_{z} with R¹ denoting a linear or branched, saturated or unsaturated alkyl and/or alkenyl residue having 2 to 30 C atoms, R² denoting hydrogen, a methyl, ethyl, propyl or isopropyl residue, R³ denoting a linear or branched alkyl residue having 2 to 30 C atoms, x denoting 0 or a number from 1 to 20, Y a number from 1 to 30 and z denoting the number 1, 2, 3, 4 or 5,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanol amides and fatty amines,
sugar surfactants of the alkyl and alkenyl oligoglycoside type,
sugar surfactants of the fatty acid-N-alkyl polyhydroxyalkylamide type,
fatty acid amide polyglycol ethers, fatty amine polyglycol ethers,
mixed ethers or mixed formulas and polysorbates.

Cationic surfactants of the formula (Tkat1-1) can additionally be used.

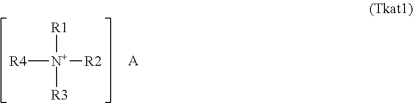

In the formula (Tkat1) R1, R2, R3 and R4 independently of one another each denote hydrogen, a methyl group, a phenyl group, a benzyl group, a saturated, branched or unbranched alkyl residue having a chain length of 8 to 30 carbon atoms, which can optionally be substituted with one or more hydroxyl groups. A denotes a physiologically acceptable anion, for example halides such as chloride or bromide and methosulfates.

Examples of compounds of the formula (Tkat1) are lauryl trimethylammonium chloride, cetyl trimethylammonium chloride, cetyl trimethylammonium bromide, cetyl trimethylammonium methosulfate, dicetyl dimethylammonium chloride, tricetyl methylammonium chloride, stearyl trimethylammonium chloride, distearyl dimethylammonium chloride, lauryl dimethyl benzylammonium chloride, behenyl trimethylammonium chloride, behenyl trimethylammonium bromide, behenyl trimethylammonium methosulfate.

The surfactants (T) are used in amounts from 0.05 to 45 wt. %, preferably 0.1 to 30 wt. % and most particularly preferably from 0.5 to 25 wt. %, relative to the total agent used according to the invention.

Emulsifiers that can be used according to the invention are for example
addition products of 4 to 30 mol of ethylene oxide and/or 0 to 5 mol of propylene oxide with linear fatty alcohols having 8 to 22 C atoms, with fatty acids having 12 to 22 C atoms and with alkylphenols having 8 to 15 C atoms in the alkyl group,
C₁₂-C₂₂ fatty acid monoesters and diesters of addition products of 1 to 30 mol of ethylene oxide with polyols having 3 to 6 carbon atoms, in particular with glycerol,
ethylene oxide and polyglycerol addition products with methyl glucoside fatty acid esters, fatty acid alkanol amides and fatty acid glucamides,
C₈-C₂₂ alkyl mono- and oligoglycosides and ethoxylated analogs thereof, wherein degrees of oligomerization of 1.1 to 5, in particular 1.2 to 2.0, and glucose as the sugar component are preferred,
mixtures of alkyl (oligo)glucosides and fatty alcohols, for example the commercially available product Montanov® 68,
addition products of 5 to 60 mol of ethylene oxide with castor oil and hydrogenated castor oil,
partial esters of polyols having 3 to 6 carbon atoms with saturated fatty acids having 8 to 22 C atoms,
sterols, from both animal tissue (zoosterols, cholesterol, lanosterol) and vegetable fats (phytosterols, ergosterol, stigmasterol, sitosterol) or from fungi and yeasts (mycosterols),
phospholipids (lecithins, phosphatidylcholines),
fatty acid esters of sugars and sugar alcohols, such as sorbitol,
polyglycerols and polyglycerol derivatives such as for example polyglycerol poly-12-hydroxystearate (commercial product Dehymuls® PGPH).

The agents according to the invention include emulsifiers preferably in amounts from 0.1 to 25 wt. %, in particular 0.5 to 15 wt. %, relative to the total agent.

The compositions according to the invention include fats (Fat) as a further active ingredient to particular advantage. Fats (Fat) are understood to be fatty acids, fatty alcohols, natural and synthetic waxes, which can be present both in solid form and in liquid form in aqueous dispersion, and natural and synthetic cosmetic oil components.

Linear and/or branched, saturated and/or unsaturated fatty acids having 6 to 30 carbon atoms can be used as fatty acids (Fatac). Fatty acids having 10 to 22 carbon atoms are preferred. Examples which can be cited include the isostearic acids, such as the commercial products Emersol® 871 and Emersol® 875, and isopalmitic acids such as the commercial product Edenor® IP 95, as well as all further fatty acids sold under the Edenor® trade names (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. The fatty acid cuts obtainable from coconut oil or palm oil are conventionally particularly preferred; as a rule the use of stearic acid is preferred in particular.

The amount used here is 0.1 to 15 wt. %, relative to the total agent. The amount is preferably 0.5 to 10 wt. %, wherein amounts of 1 to 5 wt. % can be most particularly advantageous.

Saturated, mono- or polyunsaturated, branched or unbranched fatty alcohols having C₆ to C₃₀, preferably C₁₀ to C₂₂ and most particularly preferably C₁₂ to C₂₂ carbon atoms can be used as fatty alcohols (Fatal). Suitable for use within the meaning of the invention are for example decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as the Guerbet alcohols thereof, wherein this list is intended to be of an exemplary and non-limiting nature. However, the fatty alcohols derive from preferably natural fatty acids, wherein it can conventionally be assumed that they are obtained from the esters of fatty acids by reduction. Likewise suitable for use according to the invention are fatty alcohol cuts constituting a mixture of different fatty alcohols. Such substances are available commercially for example under the names Stenol®, e.g. Stenol® 1618 or Lanette®, e.g. Lanette® 0 or Lorol®, e.g. Lorol® C8, Lorol® C14, Lorol® C18, Lorol® C₈₋₁₈, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols can of course also be used according to the invention, such as are available commercially for example under the names Corona®, White Swan®, Coronet® or Fluilan®. The fatty alcohols are used in amounts from 0.1 to 30 wt. %, relative to the total preparation, preferably in amounts from 0.1 to 20 wt. %.

Solid paraffins or isoparaffins, carnauba waxes, beeswaxes, candelilla waxes, ozokerites, ceresin, spermaceti wax, sunflower wax, fruit waxes such as for example apple wax or citrus wax, PE or PP microwaxes can be used according to the invention as natural or synthetic waxes (Fatwax). Such waxes are available for example from Kahl & Co., Trittau.

The amount used is 0.1 to 50 wt. %, relative to the total agent, preferably 0.1 to 20 wt. % and particularly preferably 0.1 to 15 wt. %, relative to the total agent.

The total amount of oil and fat components in the agents according to the invention is conventionally 0.5 to 75 wt. %, relative to the total agent. Amounts from 0.5 to 35 wt. % are preferred according to the invention.

Protein hydrolysates and/or derivatives thereof (P) are a further synergistic active ingredient according to the invention in the compositions according to the invention with the active ingredient complex according to the invention.

According to the invention protein hydrolysates of both plant and animal or marine or synthetic origin can be used.

Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Also preferred according to the invention are plant protein hydrolysates, such as for example soy, almond, pea, moring a, potato and wheat protein hydrolysates. Such products are available for example under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Sérobiologiques.

Further protein hydrolysates that are preferred according to the invention are of marine origin. They include for example collagen hydrolysates of fish or algae and protein hydrolysates of mussels or pearl hydrolysates. Examples of pearl extracts according to the invention are the commercial products Pearl Protein Extract BG® or Crodarom® Pearl.

The protein hydrolysates and derivatives thereof also include cationized protein hydrolysates, wherein the underlying protein hydrolysate can derive from animal sources, for example from collagen, milk or keratin, from plant sources, for example from wheat, corn, rice, potatoes, soy or almonds, from marine life forms, for example from fish collagen or algae, or from protein hydrolysates obtained by biotechnology. Typical examples of the cationic protein hydrolysates and derivatives according to the invention are the products that are listed under the INCI names in the “International Cosmetic Ingredient Dictionary and Handbook”, (seventh edition 1997, The Cosmetic, Toiletry, and Fragrance Association, 1101 17th Street, N.W., Suite 300, Washington, D.C. 20036-4702) and that are available commercially.

The protein hydrolysates (P) are include d in the compositions in concentrations from 0.001 wt. % to 20 wt. %, preferably from 0.05 wt. % to 15 wt. % and most particularly preferably in amounts from 0.05 wt. % to 5 wt. %.

A further preferred group of ingredients of the compositions according to the invention with the active ingredient complex according to the invention are vitamins, provitamins or vitamin precursors. Vitamins, provitamins and vitamin precursors are particularly preferred that are assigned to groups A, B, C, E, F and H.

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents according to the invention include the vitamin A component preferably in amounts from 0.05 to 1 wt. %, relative to the total preparation.

The vitamin B group or vitamin B complex includes inter alia:

Vitamin B1 (thiamine)
Vitamin B2 (riboflavin)
Vitamin B3. The compounds nicotinic acid and nicotinic acid amide (niacinamide) are often include d under this term. Preferred according to the invention is nicotinic acid amide, which is preferably include d in the agents used according to the invention in amounts from 0.05 to 1 wt. %, relative to the total agent.
Vitamin B₅ (pantothenic acid, panthenol and pantolactone). Within the context of this group panthenol and/or pantolactone is preferably used. Derivatives of panthenol which can be used according to the invention are in particular the esters and ethers of panthenol as well as cationically derivatized panthenols. Individual representatives are for example panthenol triacetate, panthenol monoethyl ether and the monoacetate thereof, and cationic panthenol derivatives. Pantothenic acid is preferably used in the present invention as a derivative in the form of the more stable calcium salts and sodium salts (Ca pantothenate, Na pantothenate).
Vitamin B6 (pyridoxine as well as pyridoxamine and pyridoxal).

The cited compounds of the vitamin B type, in particular vitamin B3, B5 and B6, are include d in the agents according to the invention preferably in amounts from 0.05 to 10 wt. %, relative to the total agent. Amounts from 0.1 to 5 wt. % are particularly preferred.

Vitamin C (ascorbic acid). Vitamin C is used in the agents according to the invention preferably in amounts from 0.1 to 3 wt. %, relative to the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular esters such as acetate, nicotinate, phosphate and succinate, are preferably include d in the agents according to the invention in amounts from 0.05 to 1 wt. %, relative to the total agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably include d in the agents according to the invention in amounts from 0.0001 to 1.0 wt. %, in particular in amounts from 0.001 to 0.01 wt. %.

The compositions according to the invention preferably include vitamins, provitamins and vitamin precursors from groups A, B, E and H. Panthenol, pantolactone, pyridoxine and derivatives thereof as well as nicotinic acid amide and biotin are particularly preferred.

A particularly preferred group of ingredients in the cosmetic compositions according to the invention is constituted by the betaines listed below: carnitine, carnitine tartrate, carnitine magnesium citrate, acetyl carnitine, betalains, 1,1-dimethyl proline, choline, choline chloride, choline bitartrate, choline dihydrogen citrate and the compound N,N,N-trimethylglycine, which is classed in the literature as betaine.

In a further embodiment that is preferred according to the invention the compositions according to the invention include bioquinones. In the agents according to the invention suitable bioquinones are understood to be one or more ubiquinones and/or plastoquinones. The ubiquinones that are preferred according to the invention have the following formula:

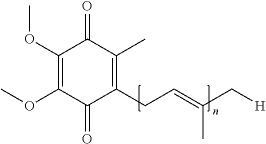

with n=6, 7, 8, 9 or 10. Coenzyme Q-10 is most preferred here.

Preferred compositions according to the invention include purine and/or purine derivatives in relatively narrow quantity ranges. Cosmetic agents that are preferred according to the invention are characterized in that they include—relative to their weight—0.001 to 2.5 wt. %, preferably 0.0025 to 1 wt. %, particularly preferably 0.005 to 0.5 wt. % and in particular 0.01 to 0.1 wt. % of purine(s) and/or purine derivative(s). Cosmetic agents that are preferred according to the invention are characterized in that they include purine, adenine, guanine, uric acid, hypoxanthine, 6-purinethiol, 6-thioguanine, xanthine, caffeine, theobromine or theophylline. In hair cosmetics preparations caffeine is most preferred.

In a further preferred embodiment of the present invention the cosmetic agent includes ectoine ((S)-2-methyl-1,4,5,6-tetrahydro-4-pyrimidine carboxylic acid). Particularly preferred according to the invention are agents which include—relative to their weight—0.00001 to 10.0 wt. %, preferably 0.0001 to 5.0 wt. % and in particular 0.001 to 3 wt. % of active ingredients from the group formed from carnitine, coenzyme Q-10, ectoine, a vitamin of the B series, a purine and derivatives or physiologically acceptable salts thereof.

A most particularly preferred care additive in the hair treatment agents according to the invention is taurine. Taurine is understood to be exclusively 2-aminoethane sulfonic acid, and a derivative to be the explicitly cited taurine derivatives. The taurine derivatives are understood to be N-monomethyl taurine, N,N-dimethyltaurine, taurine lysylate, taurine tartrate, taurine ornithate, lysyl taurine and ornithyl taurine.

Agents according to the invention which include—relative to their weight—0.0001 to 10.0 wt. %, preferably 0.0005 to 5.0 wt. %, particularly preferably 0.001 to 2.0 wt. % and in particular 0.001 to 1.0 wt. % of taurine and/or a taurine derivative are particularly preferred.

The effect of the compositions according to the invention can be further increased by means of a 2-pyrrolidinone-5-carboxylic acid and derivatives (J) thereof. The sodium, potassium, calcium, magnesium or ammonium salts are preferred, in which the ammonium ion bears one to three C₁ to C₄ alkyl groups in addition to hydrogen. The sodium salt is most particularly preferred. The amounts used in the agents according to the invention are 0.05 to 10 wt. %, relative to the total agent, particularly preferably 0.1 to 5, and in particular 0.1 to 3 wt. %.

Through the use of plant extracts as care substances the hair treatment agents according to the invention can be formulated so that they are particularly close to nature and yet very effective in their care performance. Otherwise conventional preservatives can even optionally be dispensed with. Preferred above all according to the invention are the extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, valerian, lady's smock, wild thyme, yarrow, thyme, melissa, restharrow, coltsfoot, marshmallow, meristem, ginseng, coffee, cocoa, moring a, ginger root and ayurvedic plant extracts such as for example Aegle marmelos (bilva), Cyperus rotundus (nagarmotha), Emblica officinalis (amalaki), Morinda citrifolia (ashyuka), Tinospora cordifolia (guduchi), Santalum album (chandana), Crocus sativus (kumkuma), Cinnamonum zeylanicum and Nelumbo nucifera (kamala), grasses such as wheat, barley, rye, oats, spelt, corn, the various types of millet (proso millet, finger millet, foxtail millet as examples), sugar cane, ryegrass, meadow foxtail, false oat-grass, bentgrass, meadow fescue, moor grass, bamboo, cottongrass, pennisetums, Andropogonodeae (Imperata cylindrica, also known as blood grass or cogon grass), buffalo grass, cord grass, dog's tooth grass, lovegrass, Cymbopogon (citronella grass), Oryzeae (rice), Zizania (wild rice), marram grass, blue oatgrass, soft-grass, quaking grass, speargrass, couch grass and Echinacea, in particular Echinacea purpurea (L.) Moench, all types of vine and pericarp of Litchi chinensis. The plant extracts can be used according to the invention in both pure and diluted form. If they are used in diluted form they conventionally include approximately 2 to 80 wt. % of active substance and as the solvent the extracting agent or mixture of extracting agents used to obtain them.

It can occasionally be necessary to use anionic polymers. Examples of anionic monomers which can constitute such polymers are acrylic acid, methacrylic acid, crotonic acid, maleic anhydride and 2-acrylamido-2-methylpropane sulfonic acid. Some or all of the acid groups therein can be present as the sodium, potassium, ammonium, mono- or triethanolammonium salt. Preferred monomers are 2-acrylamido-2-methylpropane sulfonic acid and acrylic acid.

Anionic polymers including as the sole monomer or as a co-monomer 2-acrylamido-2-methylpropane sulfonic acid, in which some or all of the sulfonic acid group can be present as the sodium, potassium, ammonium, mono- or triethanolammonium salt, have proved to be most particularly effective.

The homopolymer of 2-acrylamido-2-methylpropane sulfonic acid, which is available commercially for example under the name Rheothik® 11-80, is particularly preferred.

Preferred non-ionogenic monomers are acrylamide, methacrylamide, acrylic acid ester, methacrylic acid ester, vinylpyrrolidone, vinyl ether and vinyl ester.

Preferred anionic copolymers are acrylic acid-acrylamide copolymers as well as in particular polyacrylamide copolymers with monomers including sulfonic acid groups. Such a polymer is include d in the commercial product Sepigel® 305 from SEPPIC.

Likewise preferred anionic homopolymers are uncrosslinked and crosslinked polyacrylic acids. Allyl ethers of pentaerythritol, of sucrose and of propylene can be preferred crosslinking agents here. Such compounds are available commercially for example under the trademark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is available commercially under the name Stabileze® QM.

The anionic polymers are include d in the agents according to the invention preferably in amounts from 0.05 to 10 wt. %, relative to the total agent. Amounts from 0.1 to 5 wt. % are particularly preferred.

In a further embodiment the agents according to the invention can include non-ionogenic polymers.

Suitable non-ionogenic polymers are for example:
cellulose ethers, such as hydroxypropyl cellulose, hydroxyethyl cellulose and methylhydroxypropyl cellulose, such as are sold for example under the trademarks Culminal® and Benecel® (AQUALON), and Natrosol® types (Hercules);
starch and derivatives thereof, in particular starch ethers, for example Structure® XL (National Starch), a multifunctional, salt-tolerant starch;
shellac.

The non-ionic polymers are include d in the compositions according to the invention preferably in amounts from 0.05 to 10 wt. %, relative to the total agent. Amounts from 0.1 to 5 wt. % are particularly preferred.

In a further embodiment the agents according to the invention should additionally include at least one UV light screening filter. UVB filters can be oil-soluble or water-soluble.

Examples of oil-soluble substances that can be cited include:
3-benzylidene camphor, for example 3-(4-methylbenzylidene)camphor;
4-aminobenzoic acid derivatives, preferably 4-(dimethylamino)benzoic acid-2-ethylhexyl ester, 4-(dimethylamino)benzoic acid-2-octyl ester and 4-(dimethylamino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3-phenylcinnamic acid-2-ethylhexyl ester (octocrylene);
esters of salicylic acid, preferably salicylic acid-2-ethylhexyl ester, salicylic acid-4-isopropylbenzyl ester, salicylic acid homomethyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzomalonic acid di-2-ethylhexyl ester;
triazine derivatives, such as for example 2,4,6-trianilino-(p-carbo-2′-ethyl-1′-hexyloxy)-1,3,5-triazine and octyl triazone;
propane-1,3-diones, such as for example 1-(4-tert-butylphenyl)-3-(4′-methoxyphenyl)propane-1,3-dione.

Suitable water-soluble substances include:
2-phenylbenzimidazole-5-sulfonic acid and alkali, alkaline-earth, ammonium, alkyl ammonium, alkanol ammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as for example 4-(2-oxo-3-bornylidene methyl)benzenesulfonic acid and 2-methyl-5-(2-oxo-3-bornylidene)sulfonic acid and salts thereof.

Derivatives of benzoyl methane are suitable in particular as typical UV-A filters, such as for example 1-(4′-tert-butylphenyl)-3-(4′-methoxyphenyl)propane-1,3-dione or 1-phenyl-3-(4′-isopropylphenyl)propane-1,3-dione. The UV-A and UV-B filters can naturally also be used in mixtures. In addition to the cited soluble substances, insoluble pigments, in particular finely dispersed metal oxides or salts, are suitable for this purpose, such as for example titanium dioxide, zinc oxide, iron oxide, aluminum oxide, cerium oxide, zirconium oxide, silicates (talc), barium sulfate and zinc stearate. The particles should have an average diameter of less than 100 nm, preferably between 5 and 50 nm and in particular between 15 and 30 nm. They can have a spherical form, but such particles having an ellipsoid form or other form deviating from the spherical shape can also be used.

The cosmetic agents can furthermore include additional active ingredients, auxiliary substances and additives, such as for example
texturizing agents such as maleic acid and lactic acid,
swelling agents such as urea, allantoin, carbonates or hydantoin,
dimethyl isosorbide and cyclodextrins,
dyes to color the agent,
anti-dandruff active ingredients such as piroctone olamine, zinc omadine and climbazole,
complexing agents such as EDTA, NTA, β-alanine diacetic acid and phosphonic acids,
opacifiers such as latex, styrene/PVP and styrene/acrylamide copolymers,
pearlescent agents such as ethylene glycol mono- and distearate as well as PEG-3 distearate,
pigments,
stabilizing agents for hydrogen peroxide and other oxidizing agents,
propellants such as propane-butane mixtures, N₂O, dimethyl ether, CO₂ and air,
antioxidants,
perfume oils, scents and fragrances.

With regard to further optional components and to the amounts of these components used, reference is expressly made to the relevant manuals known to the person skilled in the art.

The invention therefore also provides a method for hair treatment, in which a hair treatment agent according to claim 1 is applied to the hair and is rinsed out of the hair after a contact period.

The contact period is preferably a few seconds to 100 minutes, particularly preferably 1 to 50 minutes and most particularly preferably 1 to 30 minutes.

A method in which a cosmetic agent according to claim 1 is applied to the hair and remains there is also in accordance with the invention. “Remains on the hair” is understood to mean according to the invention that the agent is not rinsed out of the hair again immediately after being applied. In this case the agent instead remains on the hair for more than 100 minutes and up to the next time the hair is washed.

Finally, the use of a composition as described above to reduce and/or delay the formation of dandruff on the scalp is according to the invention.

The compositions according to the invention are preferably formulated as a pump spray, aerosol spray, pump foam or aerosol foam.

To this end the agents according to the invention are presented in a dispensing device, which is either a compressed gas cylinder (aerosol container) additionally filled with a propellant or a non-aerosol container.

Compressed gas cylinders, with the aid of which a product is distributed through a valve via the internal gas pressure in the cylinder, are by definition termed aerosol containers. In contrast to the aerosol definition, a “non-aerosol container” is defined as a container under normal pressure, with the aid of which a product is distributed through a pump system by means of a mechanical action.

The agents according to the invention are preferably presented as an aerosol hair foam or aerosol hair spray. The agent according to the invention therefore preferably additionally includes at least one propellant.

Propellants that are suitable according to the invention are selected for example from N₂O, dimethyl ether, CO₂, air, alkanes having 3 to 5 carbon atoms, such as propane, n-butane, isobutane, n-pentane and isopentane, and mixtures thereof. Dimethyl ether, propane, n-butane, isobutane and mixtures thereof are preferred.

According to a preferred embodiment the specified alkanes, mixtures of the specified alkanes or mixtures of the specified alkanes with dimethyl ether are used as the sole propellant. However, the invention expressly also comprises the incorporation of propellants of the chlorofluorocarbon type, but in particular fluorocarbons.

In the given spray device the sizes of the aerosol droplets or foam bubbles and the size distribution in each case can be adjusted by the ratio of propellant to the other constituents of the preparations.

The amount of propellant used varies according to the specific composition of the agent, the packaging used and the desired product type, for instance hair spray or hair foam. If conventional spray devices are used, aerosol foam products preferably include the propellant in amounts from 1 to 35 wt. %, relative to the total product. Amounts from 2 to 30 wt. %, in particular from 3 to 15 wt. %, are particularly preferred. Aerosol sprays generally include larger amounts of propellant. In this case the propellant is preferably used in an amount from 30 to 98 wt. %, relative to the total product. Amounts from 40 to 95 wt. %, in particular from 50 to 95 wt. %, are particularly preferred.

The aerosol products can be produced in the conventional manner. All constituents of the individual agent with the exception of the propellant are generally introduced into a suitable pressure-resistant container. This is then closed with a valve. Finally the desired amount of propellant is added using conventional techniques.

Isopentane is preferably suitable as a propellant for expanding agents in gel form in a two-chamber aerosol container, it being incorporated into the agents according to the invention and introduced into the first chamber of the two-chamber aerosol container. At least one further propellant that is different from isopentane and that builds a higher pressure in the two-chamber aerosol container than isopentane is introduced into the second chamber of the two-chamber aerosol container. The propellants of the second chamber are preferably selected from N₂O, dimethyl ether, CO₂, air, alkanes having 3 or 4 carbon atoms (such as propane, n-butane, isobutane) and mixtures thereof.

Aerosol hair foams or aerosol hair sprays including the agent according to the invention as described above and at least one propellant are a preferred embodiment of the agents according to the invention.

Preferred agents according to the invention and propellants of the aerosol hair foam or aerosol hair spray and the amounts of propellant correspond to the details specified above.

However, highly preferred compositions according to the invention are presented as non-aerosols. As already explained, this requires special pumps and delivery systems. These are very familiar to the person skilled in the art. Known and extremely suitable systems are available for example from Airspray International BV, for example under the product name Airfoamer.

If it is presented as a non-aerosol foam in a suitable container having a suitable delivery and pump mechanism, the foam is generally produced through a fine-mesh screen in the pump head with the aid of air that is drawn in by the pump at the same time. To this end it is advantageous for the total composition according to the invention to have a viscosity from 1 to 35,000, more advantageously from 1 to 10,000, still more preferably from 1 to 5000 and most preferably from 2 to 500 mPas. The viscosity is measured by conventional measures known to the person skilled in the art.

The examples below are intended to illustrate the subject matter of the present invention without however limiting its scope.

### EXAMPLES

Unless otherwise specified, all stated amounts are parts by weight. The following formulations were prepared using known production methods.

Care spray, which can also be used in the form of a foam and/or as a hair mask:

| | K1 | K2 | K3 | K4 | K5 | K6 | K7 | K8 | K9 | K10 | K11 |
|---|---|---|---|---|---|---|---|---|---|---|---|
| Polymer JR 400 | 0.2 | 0.2 | 0.2 | 0.2 | 0.2 | 0.2 | 0.2 | 0.2 | 0.2 | 0.2 | 0.2 |
| Armocare VGH 70 | 0.5 | 0.5 | 0.5 | 0.5 | 0.5 | 0.5 | 0.5 | 0.5 | 0.5 | 0.5 | 0.5 |
| Stearamidopropyl | 0.5 | 0.5 | 0.5 | 0.5 | 0.5 | 0.5 | 0.5 | 0.5 | 0.5 | 0.5 | 0.5 |
| dimethylamine | | | | | | | | | | | |
| PVP/VA copolymer 60/40 | 1.0 | 1.0 | 1.0 | 1.0 | 1.0 | 1.0 | 1.0 | 1.0 | 1.0 | 1.0 | 1.0 |
| Polyquaternium-74 | 0.5 | — | — | — | — | — | — | — | — | — | — |
| Polyquaternium-69 | — | 0.5 | — | — | — | — | — | — | — | 0.5 | — |
| Polyquaternium-39 | — | — | 0.5 | — | — | — | — | — | — | — | — |
| Polyquaternium-72 | — | — | — | 0.5 | — | — | — | — | — | 0.3 | 0.3 |
| Polyquaternium-16 | — | — | — | — | 0.5 | — | — | — | — | — | — |
| Polyquaternium-55 | — | — | — | — | — | 0.5 | — | — | — | — | — |
| Polyquaternium-44 | — | — | — | — | — | — | 0.5 | — | — | — | — |
| Polyquaternium-68 | — | — | — | — | — | — | — | 0.5 | — | — | — |
| Polyquaternium-89 | — | — | — | — | — | — | — | — | 0.5 | — | 0.3 |
| Panthenol | 0.2 | 0.2 | 0.2 | 0.2 | 0.2 | 0.2 | 0.2 | 0.2 | 0.2 | 0.2 | 0.2 |
| Cetrimonium chloride | 5.0 | 5.0 | 5.0 | 5.0 | 5.0 | 5.0 | 5.0 | 5.0 | 5.0 | 5.0 | 5.0 |
| Ceteareth-25 | 0.3 | 0.3 | 0.3 | 0.3 | 0.3 | 0.3 | 0.3 | 0.3 | 0.3 | 0.3 | 0.3 |
| Protein hydrolysate | 0.2 | 0.2 | 0.2 | 0.2 | 0.2 | 0.2 | 0.2 | 0.2 | 0.2 | 0.2 | 0.2 |
| Dow Corning 193 | 0.2 | 0.2 | 0.2 | 0.2 | 0.2 | 0.2 | 0.2 | 0.2 | 0.2 | 0.2 | 0.2 |
| Silicone Quaternium-22 | 0.5 | 0.5 | 0.5 | 0.5 | 0.5 | 0.5 | 0.5 | 0.5 | 0.5 | 0.5 | 0.5 |
| Coco betaine | 0.5 | 0.5 | 0.5 | 0.5 | 0.5 | 0.5 | 0.5 | 0.5 | 0.5 | 0.5 | 0.5 |
| Water, preservatives and | to 100 | to 100 | to 100 | to 100 | to 100 | to 100 | to 100 | to 100 | to 100 | to 100 | to 100 |
| optionally perfume oils | | | | | | | | | | | |

The pH values of all formulations were set to 2 to 6.

For application as a foam, the formulation in question is either introduced into an aerosol container with a propellant or dispensed as a foam from a pump bottle with a corresponding pump attachment, such as an Airfoamer for example.

For application as a hair mask or cream, fatty alcohols such as cetyl stearyl alcohol and/or ethylene glycol distearate and/or glycerol monostearate are added to the above formulations in amounts from 0.2 to 5.0 wt. %.

Shampoo:

| | S1 | S2 | S3 | S4 | S5 | S6 | S7 | S8 | S9 | S10 | S11 |
|---|---|---|---|---|---|---|---|---|---|---|---|
| Texapon ® N70 | 15.0 | 15.0 | 15.0 | 15.0 | 15.0 | 15.0 | 15.0 | 15.0 | 15.0 | 15.0 | 15.0 |
| Arlypon ® F | 0.15 | 0.15 | 0.15 | 0.15 | 0.15 | 0.15 | 0.15 | 0.15 | 0.15 | 0.15 | 0.15 |
| Antil ® 141 | 0.15 | 0.15 | 0.15 | 0.15 | 0.15 | 0.15 | 0.15 | 0.15 | 0.15 | 0.15 | 0.15 |
| Disodium | 6.0 | 6.0 | 6.0 | 6.0 | 6.0 | 6.0 | 6.0 | 6.0 | 6.0 | 6.0 | 6.0 |
| cocoamphodiacetate | | | | | | | | | | | |
| Polyquaternium-74 | 0.5 | — | — | — | — | — | — | — | — | — | — |
| Polyquaternium-69 | — | 0.5 | — | — | — | — | — | — | — | 0.5 | — |
| Polyquaternium-39 | — | — | 0.5 | — | — | — | — | — | — | — | — |
| Polyquaternium-72 | — | — | — | 0.5 | — | — | — | — | — | 0.3 | 0.3 |
| Polyquaternium-16 | — | — | — | — | 0.5 | — | — | — | — | — | — |
| Polyquaternium-55 | — | — | — | — | — | 0.5 | — | — | — | — | — |
| Polyquaternium-44 | — | — | — | — | — | — | 0.5 | — | — | — | — |
| Polyquaternium-68 | — | — | — | — | — | — | — | 0.5 | — | — | — |
| Polyquaternium-89 | — | — | — | — | — | — | — | — | 0.5 | — | 0.3 |
| Cetiol ® HE | 3.0 | 3.0 | 3.0 | 3.0 | 3.0 | 3.0 | 3.0 | 3.0 | 3.0 | 3.0 | 3.0 |
| Panthenol | 0.2 | 0.2 | 0.2 | 0.2 | 0.2 | 0.2 | 0.2 | 0.2 | 0.2 | 0.2 | 0.2 |
| Dow Corning ® 193 | 0.1 | 0.1 | 0.1 | 0.1 | 0.1 | 0.1 | 0.1 | 0.1 | 0.1 | 0.1 | 0.1 |
| Silicone Quaternium- | 1.0 | 1.0 | 1.0 | 1.0 | 1.0 | 1.0 | 1.0 | 1.0 | 1.0 | 1.0 | 1.0 |
| 22 | | | | | | | | | | | |
| Protein hydrolysate | 0.5 | 0.5 | 0.5 | 0.5 | 0.5 | 0.5 | 0.5 | 0.5 | 0.5 | 0.5 | 0.5 |
| Cremophor ® HRE 60 | 0.5 | 0.5 | 0.5 | 0.5 | 0.5 | 0.5 | 0.5 | 0.5 | 0.5 | 0.5 | 0.5 |
| Water, preservatives | to 100 | to 100 | to 100 | to 100 | to 100 | to 100 | to 100 | to 100 | to 100 | to 100 | to 100 |
| and optionally perfume | | | | | | | | | | | |
| oils | | | | | | | | | | | |

The pH values of all formulations were set to 4.5 to 5.8.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent including in a suitable cosmetic carrier—relative in each case to the total composition of the agent—
a) at least one non-ionic polymer in a total amount from 0.01 to 10.0 wt. %, selected from the non-ionic polymers including at least one structural unit selected from the group of structural units of formulae (M1) to (M3) 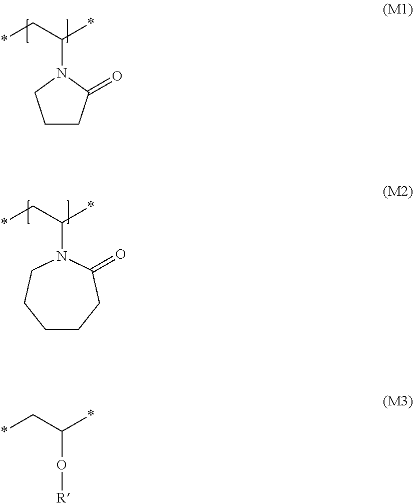
in which R′ denotes a hydrogen atom or a (C₂ to C₁₈) acyl group
and
b) at least one cationic amino silicone having at least three terminal amino-functional groups in a total amount from 0.01 to 5.0 wt. %.

2. The hair treatment agent according to claim 1, characterized in that moreover at least one surfactant selected from the zwitterionic and/or amphoteric surfactants is include d in a total amount from 0.01 to 5.0 wt. %.

3. The hair treatment agent according to claim 1, characterized in that moreover at least one quaternary ammonium compound is include d in a total amount from 0.1 to 10.0 wt. %, relative to the weight of the total composition, selected from at least one of the groups of
i) esterquats,
ii) quaternary imidazolines of formula (Tkat2), 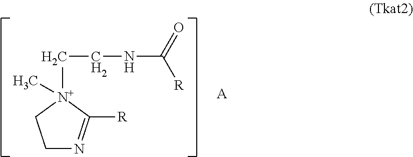
in which the residues R independently of one another each denote a saturated or unsaturated, linear or branched hydrocarbon residue having a chain length of 8 to 30 carbon atoms and A denotes a physiologically acceptable anion, and/or
iii) amines and/or cationized amines,
iv) poly(methacryloyloxyethyltrimethylammonium) compounds,
v) quaternized cellulose derivatives,
vi) cationic alkyl polyglycosides,
vii) cationized honey,
viii) cationic guar derivatives,
ix) chitosan,
x) polymeric dimethyldiallyl ammonium salts and copolymers thereof with esters and amides of acrylic acid and methacrylic acid,
xi) copolymers of vinylpyrrolidone with quaternized derivatives of dialkylaminoalkyl acrylate and methacrylate,
xii) vinylpyrrolidone-vinylimidazolium methochloride copolymers,
xiii) quaternized polyvinyl alcohol, and
xiv) Polyquatemium-74,
and mixtures thereof.

4. The hair treatment agent according to claim 1, characterized in that the cationic amino silicone is selected from the group consisting of Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quatemium-8, Silicone Quatemium-9, Silicone Quatemium-10, Silicone Quaternium-11, Silicone Quatemium-12, Silicone Quaternium-15, Silicone Quaternium-16, Silicone Quatemium-17, Silicone Quaternium-18, Silicone Quaternium-20, Silicone Quatemium-21 and Silicone Quaternium-22.

5. The hair treatment agent according to claim 1, wherein the cationic amino silicone is Silicone Quaternium-22.

6. The hair treatment agent according to claim 2, wherein the zwitterionic and/or amphoteric surfactant is selected from cocamidopropyl betaine and/or coco betaine.

7. The hair treatment agent according to claim 1, wherein the non-ionic polymer is a copolymer of N-vinylpyrrolidone and vinyl esters of carboxylic acids having 2 to 18 carbon atoms.

8. The hair treatment agent according to claim 1, further comprising at least one active ingredient selected from the group consisting of carnitine, taurine, coenzyme Q-10, ectoine, a purine and derivatives thereof, and a vitamin of the B series.

9. The hair treatment agent according to claim 3, wherein the quaternary ammonium compound is selected from the group consisting of stearamidopropyl dimethylamine, distearoylethyl hydroxyethylmonium methosulfate, dicocoyl hydroxyethylmonium methosulfate, dipalmitoylethyldimonium chloride, Quaternium-27, Quaternium-91 and behenoyl PG trimonium chloride.

10. A method for treating keratinic fibers, comprising:
applying a cosmetic composition according to claim 1 to the keratinic fibers, and
rinsing the cosmetic composition out after a contact time of a few seconds up to 45 minutes.
